SEQUENCE LISTING (2 sequences)

SEQ ID NO: 1 - DNA, 22 bp, Artificial Sequence
  Description of Artificial Sequence Synthetic oligonucleotide
gcggttgtggttatcttctgag

SEQ ID NO: 2 - DNA, 23 bp, Artificial Sequence
  Description of Artificial Sequence Synthetic oligonucleotide
cccaaagttcacattccacttca

(19) United States
(12) Patent Application Publication
(10) Pub. No.: US 2011/0230448 A1
(43) Pub. Date: Sep. 22, 2011

(54) Treatment and/or Prevention of Non-Viral Epithelial Damage

(75) Inventor: Gerard BRADY, Charlesworth (GB)

(73) Assignee: Epistem Limited, Manchester (GB)

(21) Appl. No.: 13/073,605

(22) Filed: Mar. 28, 2011

Related U.S. Application Data
(63) Continuation of application No. 12/469,259, filed on May 20, 2009, now abandoned.
Application No. 12/469,259 is a continuation of application No. 10/547,650, filed on Sep. 1, 2005, now abandoned.

(30) Foreign Application Priority Data
Mar. 14, 2003 (GB) 0305835.1
Mar. 14, 2003 (GB) 0305837.7

(51) Int. Cl. A61K 31/662; C07F 9/38; C07K 14/50; C07K 14/47; A61P 17/00; A61P 1/00; A61P 1/12; A61P 1/04; A61P 29/00; A61K 31/513

(52) U.S. Cl. 514/120; 562/24; 530/399; 530/350; 514/274

(57) ABSTRACT

There is provided the use of an inhibitor of phosphate transporter activity for the manufacture of a medicament for the prevention and/or treatment of non-viral damage to an epithelium, or of a condition caused or characterised by such damage. The inhibitor of phosphate transporter activity may optionally be a phosphono-carboxylic acid, or a pharmaceutically acceptable derivative of such an acid. There are also provided methods of treatment using such inhibitors, acids and derivatives.

The present invention relates to medicaments for the treatment and/or prevention of non-viral epithelial damage. It also provides methods of treatment and/or prevention of non-viral epithelial damage.

Epithelial layers are found throughout the body where they fulfil a number of roles, including mechanical protection and active transport associated with e.g. homeostasis or food uptake. Examples of epithelial tissues include the epidermis of the skin (such as the scalp), and the linings of the digestive system, lungs and blood vessels.

Epithelia are frequently subject to non-viral damage that impairs the normal function of the epithelium. Such non-viral damage may arise in a wide range of manners, including through mechanical injury, by action of infectious agents such as bacteria and fungi, through cytotoxic chemical agents, or via other sources of damage, such as radiation damage.

In general epithelial tissues share many features in common. For example, studies on the gastrointestinal epithelium have previously proved to be good indicators of damage response and regeneration processes in other epithelia (Potten, C. S. (1991). Regeneration in epithelial proliferative units as exemplified by small intestinal crypts. Ciba Found Symp 160, 54-71; discussion 71-56). The effects of non-viral damage vary, and are dependent on the nature of the epithelial layer damaged. For example, damage to the digestive system may cause conditions such as diarrhoea, mucositis and colitis whereas damage to the epithelium of the scalp may cause hair loss (alopecia).

Cancer therapies such as chemotherapy and radiotherapy represent a common cause of injury to the epithelia. Since these iatrogenic conditions occur as a result of elective treatment they represent particularly suitable targets for therapies designed to prevent or treat (i) epithelial damage and/or (ii) conditions caused or characterised by such damage. Furthermore, such therapies which are capable of alleviating damage to epithelia are suitable for treatment of epithelial tissues damaged by unplanned or accidental exposure to harmful stimuli.

Currently there are limited medicaments available for the prevention and/or treatment of non-viral epithelial damage and conditions caused or characterised by such damage. There is thus a recognised need to develop suitable new medicaments.

According to a first aspect of the invention there is provided the use of an inhibitor of phosphate transporter activity for the manufacture of a medicament for the prevention and/or treatment of non-viral damage to an epithelium, or a condition caused or characterised by such damage.

Inorganic phosphate is required as a critical cell nutrient for many cellular processes namely, cellular metabolism, signal transduction, lipid synthesis and regulation of enzymatic activities (Kavanaugh, M. P., Miller, D. G., Zhang, W., Law, W., Kozak, S. L., Kabat, D., and Miller, A. D. (1994). Cell-surface receptors for gibbon ape leukemia virus and amphotropic murine retrovirus are inducible sodium-dependent phosphate symporters. Proc. Natl. Acad. Sci. USA 91, 7071-7075). Inorganic phosphate can enter cells both through a passive process and also via a carrier-mediated process through phosphate transporters. Disturbance of phosphate homeostasis may effect the function of a number of cellular process and thus the roles of phosphate transporters has been the subject of several recent studies (Bottger, P., and Pedersen, L. (2002). Two highly conserved glutamate residues critical for type III sodium-dependent phosphate transport revealed by uncoupling transport function from retroviral receptor function. J Biol Chem 277, 42741-42747).

The invention is based on the finding that administration of medicaments comprising an inhibitor of phosphate transporter activity can be used to prevent and/or treat non-viral damage to an epithelium, and/or to prevent and/or treat conditions caused or characterised by non-viral epithelial damage. The treatment according to the invention may be used prophylactically to prevent non-viral damage to an epithelium, or conditions caused or characterised by such damage, or as a treatment for existing non-viral epithelial damage or conditions caused or characterised by such damage. For example, when an individual is to be exposed to an agent known to induce epithelial damage, such as many chemotherapeutic agents, the treatment according to the invention advantageously may be administered prior to, or at the same time as, the damage-inducing agent, and preferably in combination with the damage-inducing agent.

The ability of an agent to inhibit phosphate transporter activity may be assessed by established assays such as phosphate uptake assays. An example of one such assay is described by Kavanaugh and colleagues (Kavanaugh, M. P., Miller, D. G., Zhang, W., Law, W., Kozak, S. L., Kabat, D., and Miller, A. D. (1994). Cell-surface receptors for gibbon ape leukemia virus and amphotropic murine retrovirus are inducible sodium-dependent phosphate symporters. Proc. Natl. Acad. Sci. USA 91, 7071-7075). In brief, a suitable cell line is incubated with a range of known phosphate concentrations which include a proportion of radioactive phosphate. Following incubation at 37° C. for a fixed time, such as twenty minutes, cells are washed with phosphate buffered saline and the extent of radioactive phosphate uptake determined by scintillation spectroscopy.

The inhibitor of phosphate transporter activity is preferably an inhibitor of sodium-dependent phosphate transporter activity, and more preferably a specific inhibitor of sodium-dependent phosphate transporter activity. There are four known groups of sodium-phosphate co-transporter proteins: Type I, Type IIa, Type IIb, (mostly confined to the kidney) and Type III transporters (which possess a more ubiquitous tissue expression pattern). Type III transporters are predicted to have 10 membrane spanning domains with a large hydrophilic domain near the centre of each molecule, believed to be the pore-forming region of the transport protein. Members of the type I and II groups are predicted to possess only 6-8 such membrane spanning regions with a central pore region. Type III transporters share less that 20% amino acid identity with the Type I and II groups (described in Fernandes, I., Beliveau, R., Friedlander, G., and Silve, C. (1999). NaPO(4) cotransport type III (PiT1) expression in human embryonic kidney cells and regulation by PTH. Am J Physiol 277, F543-551).

The Type III group includes the phosphate transporter proteins Pit1 (also designated Gibbon leukemia virus receptor or Glvr1) and Pit2 (also designated murine amphotropic retrovirus receptor or Glvr2 or Ram2) (Kavanaugh, M. P., Miller, D. G., Zhang, W., Law, W., Kozak, S. L., Kabat, D., and Miller, A. D. 1994). Cell-surface receptors for gibbon ape leukemia virus and amphotropic murine retrovirus are inducible sodium-dependent phosphate symporters. Proc Natl Acad Sci USA 91, 7071-7075. Olah, Z., Lehel, C., Anderson, W. B., Eiden, M. V., and Wilson, C. A. (1994). The cellular receptor for gibbon ape leukemia virus is a novel high affinity sodium-dependent phosphate transporter. J Biol Chem 269, 25426-25431). Pit1 and Pit2 share approximately 60% amino acid sequence similarity.

Preferably the inhibitor is a inhibitor of type III sodium-dependent phosphate transporter activity, more preferably a specific inhibitor. Most preferably the inhibitor is a specific inhibitor of Pit 1 and/or Pit 2.

Many different types of inhibitors of phosphate transporter activity are known. For example there exists a range of chemical inhibitors of phosphate transporter activity suitable for use according to the invention. Preferred examples of such inhibitors include phosphono-carboxylic acids, and pharmaceutically acceptable derivatives of such acids, such as salts or esters.

So effective are phosphono-carboxylic acids and their derivatives that, in accordance with a second aspect of the invention there is provided the use of a phosphono-carboxylic acid, or a pharmaceutically acceptable derivative thereof, for the manufacture of a medicament for the prevention and/or treatment of non-viral damage to an epithelium, or a condition caused or characterised by such damage

The phosphono-carboxylic acids for use in accordance with the invention may be of the formula R¹R²P(O)-Lₙ-CO₂H, wherein n is 0 or 1, R¹ and R² are the same or different and are either a hydroxy group of an ester residue, and L is a hydrocarbon group having a maximum of 8 carbon atoms. Pharmaceutically acceptable derivatives, e.g. salt and esters, of these acids may also be used in accordance with the invention.

L may be an aliphatic, alicyclic or aromatic group having an all-carbon backbone. Examples of such groups include alkylene, cycloalkylene, cycloalkenylene, phenylene, alkenylene, alkynylene, cycloalkylalkylene, cycloalkenylalkylene, and phenylalkylene, phenylalkenylene and phenylalkynylene groups.

If L is an alicyclic hydrocarbon group then it will preferably include up to 7 carbon atoms in the ring, more preferably up to 6 carbon atoms.

For the case where L is an aromatic group then it is most preferably a benzene nucleus.

Alkylene groups, as examples of L, may be straight or branched and preferably have 1 to 6 carbon atoms, more preferably 1 to 4 carbon atoms. The alkaline group may for example by methylene, ethylidene or propylidene

Examples of cycloalkylene groups that my be used or any having 3 to 8 ring carbon atoms, e.g. cyclopropylidene, cyclobutylidene or any similar divalent group having 5, 6, 7 or 8 carbon atoms in the ring.

Examples of alkenylene groups are those derived from, ethene, 1-propene, 2-propene, isopropene, butene, buta-1,4-diene, pentene, and hexene.

Examples of cycloalkenylene groups include, but are not limited to, those derived from cyclopropene, cyclobutene, cyclopentene, cyclopentadiene and cyclohexene.

Examples of alkynylene groups are those having from 2 to 8 carbon atoms, more typically from 2 to 6 carbon atoms, for example from 2 to 4 carbon atoms. Examples of alkynylene groups include, but are not limited to, those derived from ethyne (acetylene) and 2-propyne groups.

The group L can be optionally substituted by one or more substituents. Examples of substituents include hydrocarbon groups having up to 4 carbon atoms, for example alkyl groups such as methyl or ethyl. Further examples of substituents include halogen atoms, for example one or more halogens selected from fluorine, chlorine and bromine.

The groups R¹ and R² are the same or different and each is hydroxy or an ester residue. Examples of ester residues are aralkyl and alkyl ester groups such as benzyloxy and alkoxy groups, a particular example being ethoxy.

In one preferred group of compounds, R¹ and R² are the same and are both hydroxyl.

Preferred Examples of phosphono carboxylic acids for use in accordance with the invention are either those in which n is 0 or n is 1 and L is a C₁₋₄ alkaline group. If n is 1 then L is preferably a methylene or an ethylidene group.

Particular preferred phosphono carboxylic acids are phosphono-formic acid, phosphono-acetic acid and α-chloro-α-bromo-phosphonoacetic acid.

Salts of esters of the phosphono carboxylic acid may also be used.

The phosphono-carboxylic acid phosphate transport inhibitor compounds of the invention can be in the form of the free acid or a salt or ester thereof.

The salt can be any pharmaceutically acceptable salt formed with a pharmaceutically acceptable cation. Examples of salts include alkaline and alkaline earth metal salts, transition metal salts and substituted or unsubstituted ammonium salts. Particular metal salts include those such as Li, Na, K, Ca, Mg, Zn, Mn and Ba salts, sodium being one preferred particular example. The salt (e.g. an alkali metal salt such as a sodium salt) can be for example a di-salt or tri-salt (e.g. di-sodium or tri-sodium salt), tri-sodium salts being preferred. Examples of ammonium salts include those formed with ammonia itself, or with primary, secondary, tertiary or quaternary amines.

Particular examples of ammonium salts include those formed with a salt forming component such as NH₃, CH₃NH₂, C₂H₅NH₂, C₃H₇NH₂, C₄H₉NH₂, C₅H₁₁NH₂, C₆H₁₃NH₂, (CH₃)₂NH, (C₂H₅)₂NH, (C₃H7)₂NH, (C₄H₉)₂NH, (C₅H₁₁)₂NH, (C₆H₁₃)₂NH, (CH₃)₃N, (C₂H₅)₃N, (C₃H₇)₃N, (C₄H₉)₃N, (C₅H₁₁)₃N, (C₆H₁₃)₃N, C₆H₅CH₂NH₂, HOCH₂CH₂NH₂, (HOCH₂CH₂)₂NH, (HOCH₂CH₂)₃N, C₂H₅NH(CH₂CH₂OH), C₂H₅N(CH₂CH₂OH)₂, (HOH₂C)₃CNH₂, piperidine, pyrrolidine and morpholine.

Examples of quaternary ammonium salts are those formed with quaternary ammonium ions such as (CH₃)₄N, (C₂H₅)₄N, (C₃H₇)₄N, (C₄H₉)₄N, (C₅H₁₁)₄N, (C₆H₁₃)₄N and C₂H₅N(CH₂CH₂OH)₃.

Where the compound is in the form of a carboxylate ester, the ester can be, for example an substituted or unsubstituted aralkyl ester such as a benzyl ester, or an alkyl ester, e.g. a C₁₋₄ alkyl ester such as an ethyl ester.

Preferred phosphono-carboxylic acids for use according to the invention include phosphonoformic acid and phosphonoacetic acid, and pharmaceutically acceptable derivatives of these acids (Swaan, P. W., and Tukker, J. J. (1995). Carrier-mediated transport mechanism of foscarnet (trisodium phosphonoformate hexahydrate) in rat intestinal tissue. J Pharmacol Exp Ther 272, 242-247. Szczepanska-Konkel, M., Yusufi, A. N., VanScoy, M., Webster, S. K., and Dousa, T. P. (1986). Phosphonocarboxylic acids as specific inhibitors of Na+-dependent transport of phosphate across renal brush border membrane. J Biol Chem 261, 6375-6383. Tsuji, A., and Tamai, I. (1989). Na⁺ and pH dependent transport of foscarnet via the phosphate carrier system across intestinal brush-border membrane. Biochem Pharmacol 38, 1019-1022). Derivatives of phosphonoformic or phosphonoacetic acid that are suitable for use in accordance with the invention include pharmaceutically acceptable salts of the acids. Generally it is preferred to use an alkali metal salt of phosphonoformic acid or phosphonoacetic acid, more particularly the sodium salt. The sodium salt may be the di or trisodium salt. Most particularly it is preferred to use the trisodium salt.

Alternatively, the salt may be the mono, di or tri ammonium salt, the primary secondary or tertiary amine salts or the quaternary ammonium salt as disclosed in Example 2 of U.S. Pat. No. 4,215,113.

A preferred derivative of a phosphono-carboxylic acid may be phosphonoformic acid trisodium salt hexahydrate, designated by its generic name foscarnet, which is a well known anti-viral agent.

A further example of a particularly preferred derivative of a phosphono-carboxylic acid is alpha-Cl-alpha-Br-phosphonoacetate, which possesses threefold greater inhibitory activity than phosphonoformic acid (Hoppe, A., McKenna, C. E., Harutunian, V., Levy, J. N., and Dousa, T. P. (1988). alpha-Cl-alpha-Br-phosphonoacetic acid is a potent and selective inhibitor of Na+/Pi cotransport across renal cortical brush border membrane. Biochem Biophys. Res Commun 153, 1152-1158). All these agents are structurally similar to the phosphate molecule and therefore act as competitive inhibitors of the sodium-dependent phosphate co-transporter system.

Inhibitors of phosphate transporter activity suitable for use in accordance with the invention may include substances capable of directly or indirectly regulating serum phosphate levels. The physiological regulators of serum phosphate have recently been collectively termed the “phosphatonins”, and such regulators represent preferred inhibitors of phosphate transporter activity for use in accordance with the invention. Examples of suitable phosphatonins that may be used include fibroblast growth factor 23 (FGF23) and secreted frizzled-related protein 4 (FRP4) (Schiavi, S. C. and R. Kumar (2004). “The phosphatonin pathway: New insights in phosphate homeostasis.” Kidney Int 65(1): 1-14).

Suitable inhibitors of phosphate transporter activity for use according to the invention may also include agents capable of interfering with the activity of phosphate transporter proteins. Such agents include chemical and protein antagonists of such transporters, including agents such as neutralising antibodies that may “block” transporter protein activity.

It will be appreciated that the inhibition of phosphate transporter activity may also be brought about by a reducing the number of phosphate transporter proteins expressed by cells of the epithelium. A suitable reduction may be brought about by reducing transcription of genes encoding phosphate transporter proteins, or by reducing translation of mRNA produced by such transcription. Agents suitable for achieving inhibition in this manner include specific inhibitors of gene expression, anti-sense oligonucleotides, anti-sense mRNA or oligonucleotides, RNAi and gene-specific ribozymes (Wang, H., Hang, J., Shi, Z., Li, M., Yu, D., Kandimalla, E. R., Agrawal, S., and Zhang, R. (2002). Antisense oligonucleotide targeted to RIalpha subunit of cAMP-dependent protein kinase (GEM231) enhances therapeutic effectiveness of cancer chemotherapeutic agent irinotecan in nude mice bearing human cancer xenografts: in vivo synergistic activity, pharmacokinetics and host toxicity. Int J Oncol 21, 73-80; Song, E., Lee, S. K., Wang, J., Ince, N., Ouyang, N., Min, J., Chen, J., Shankar, P., and Lieberman, J. (2003). RNA interference targeting Fas protects mice from fulminant hepatitis. Nat Med 9, 347-351; Abounader, R., Lal, B., Luddy, C., Koe, G., Davidson, B., Rosen, E. M., and Laterra, J. (2002). In vivo targeting of SF/HGF and c-met expression via U1snRNA/ribozymes inhibits glioma growth and angiogenesis and promotes apoptosis. Faseb J 16, 108-110). Alternatively, approaches which disrupt the regulatory pathways responsible for controlling phosphate transporter expression may also be used.

Inhibitors capable of interfering with the activity of phosphate transporters may be “directly” administered (i.e. administration of the inhibitor itself) by medicaments manufactured in accordance with the invention. Alternatively, or in addition, such inhibitors may be administered “indirectly”, for instance by administration of vector comprising a vehicle encoding a suitable inhibitor. Such a vehicle may, for instance, comprise a nucleic acid encoding a product capable of disrupting the regulatory pathways responsible for controlling phosphate transporter expression. For example, the vehicle may comprise a gene encoding an inhibitor of phosphate transporter transcription.

The skilled person will appreciate that inhibitors of phosphate transporter activity and/or phosphono-carboxylic acids (or pharmaceutically acceptable derivatives thereof) may also be used in methods to prevent and/or treat non-viral epithelial damage or a condition caused or characterised by such damage.

Thus, according to a third aspect of the invention, there is provided a method of preventing and/or treating non-viral damage to an epithelium, or a condition caused or characterised by such damage, the method comprising administering to a patient in need of such prevention and/or treatment an effective amount of an inhibitor of phosphate transporter activity.

Further, in accordance with a fourth aspect of the invention, there is provided a method of preventing and/or treating non-viral damage to an epithelium, or a condition caused or characterised by such damage, the method comprising administering to a patient in need of such prevention and/or treatment an effective amount of a phosphono-carboxylic acid, or a pharmaceutically acceptable derivative thereof.

The use of medicaments or methods of treatment in accordance with the invention is particularly suitable for the prevention and/or treatment of non-viral damage to clonogenic stem cells of the epithelium, and the prevention and/or treatment of conditions caused or characterised by such damage. The effect of the medicaments may be to protect clonogenic stem cells from damage (i.e. prevent the damage occurring), or to improve the clonogenic stem cells' ability to recover from damage (i.e. improve cell survival after damage), or a combination of these two modes of action.

The medicaments and methods of treatment of the invention are suitable for the prevention and/or treatment of epithelial damage, or conditions caused or characterised by such damage in all epithelial tissues tested. The inventors believe that the medicaments and methods of treatment of the invention may be effectively used to treat epithelial damage arising as a result of organ transplants and tissue grafting (including damage to both recipient epithelial cells and donor epithelial cells) as well as epithelial damage associated with wounds to epithelial tissues, and diseases such as psoriasis and alopecia. The use of inhibitors of phosphate transporter activity and phosphono-carboxylic acids (and their derivatives) also has utility in methods of tissue and cell culture.

We have found that the use of medicaments or methods of treatment in accordance with the invention is particularly suitable for use in the prevention and/or treatment of conditions caused or characterised by non-viral damage to digestive epithelia. By “digestive epithelia” is meant the epithelia of any tissue involved in digestion, particularly epithelia of the gastrointestinal tract (for the purposes of the present specification defined as the tract running from mouth to anus including all oral mucosa). More preferably the digestive epithelia may be the epithelium of the intestine, or the epithelium of the oral mucosa.

The medicaments and methods of treatment of the invention are also suitable for the prevention and/or treatment of non-viral damage (or conditions caused or characterised by such damage) to the epidermis, including epidermal appendages such as hair follicles. Medicaments and treatments of the invention may be used to prevent and/or treat epidermal damage such as sun burn, and associated blistering, caused by solar radiation.

In the case of damage to digestive epithelia, medicaments or methods of treatment in accordance with the invention may be used to prevent and/or treat conditions such as diarrhoea, colitis, ulcerative colitis, mucositis, ulcers, surgical or accidental wounds and reactive diseases such as inflammatory bowel disease (for example, Crohn's disease and the like).

The inventors have found that the medicaments and or methods of treatment in accordance with the invention are particularly effective for the treatment and/or prevention of epithelial damage caused by therapies employed in cancer treatment, specifically chemotherapy and radiotherapy, and the treatment and/or prevention of conditions caused or characterised by such epithelial damage.

Cancer represents the second most common cause of mortality in most developed countries. It is estimated that one in three Americans presently alive will ultimately develop cancer. Chemotherapy and radiotherapy are among the most common treatments for cancer, however it is recognised that they have many adverse side-effects. Among these side-effects there exist a number that are caused by damage inflicted on healthy epithelial cells. Commonly occurring examples include diarrhoea caused by damage to the digestive epithelia and alopecia caused by damage to epithelial cells of hair follicles found in skin such as that of the scalp. The medicaments and or methods of treatment in accordance with the invention are particularly useful for prevention and or treatment of diarrhoea caused by radiotherapy or chemotherapy administered to cancer patients.

Therefore, according to a fifth aspect of the invention there is provided the use of an inhibitor of phosphate transporter activity for the manufacture of a medicament for the prevention and/or treatment of mucositis and/or diarrhoea caused by radiotherapy and/or by chemotherapy.

In a sixth aspect of the invention there is provided the use of a phosphono-carboxylic acid, or pharmaceutically acceptable derivative thereof, for the manufacture of a medicament for the prevention and/or treatment of mucositis and/or diarrhoea caused by radiotherapy and/or by chemotherapy.

In a seventh aspect of the invention there is provided a method of preventing and/or treating mucositis and/or diarrhoea caused by radiotherapy and/or by chemotherapy, the method comprising administering to a patient in need of such prevention and/or treatment an effective amount of an inhibitor of phosphate transporter activity.

In a eighth aspect of the invention there is provided a method of preventing and/or treating mucositis and/or diarrhoea caused by radiotherapy and/or by chemotherapy, the method comprising administering to a patient in need of such prevention and/or treatment an effective amount of a phosphono-carboxylic acid, or a pharmaceutically acceptable derivative thereof.

In addition to damage to digestive epithelia and subsequent diarrhoea caused by cancer therapies, gastrointestinal damage and/or diarrhoea also frequently occur through microbial infection.

Accordingly, mucositis and/or diarrhoea caused by non-viral microbes constitute preferred conditions that may be treated and or prevented in accordance with the invention.

Non-viral microbes that may cause damage to the gastrointestinal epithelia include bacteria and fungi. Specific examples of microbes known to contribute to epithelial damage leading to conditions such as diarrhoea include Bacillus cereus, Campylobacter, Clostridium botulinum, Clostridium perfringens, Cryptosporidium parvum, Escherichia coli (including Escherichia coli O157:H7 and Escherichia coli non-O157 shiga toxin-producing, also known as STEC), Giardia intestinalis, Listeria monocytogenes, Mycobacterium bovis, Salmonella typhi and non-typhoid, Shigella (including Shigella dysenteriae), Staphylococcus aureus, Toxoplasma gondii, Vibrio cholerae, Vibrio parahaemolyticus, Vibrio vulnificus, and Yersinia enterolitica.

Inhibitors of phosphate transporter activity and phosphono-carboxylic acids, or pharmaceutically acceptable derivatives thereof, are preferably formulated as medicaments in accordance with the invention. The following paragraphs provide details of suitable formulations that may be used in the preparation of such medicaments. The term “active agent” as used in the following paragraphs is taken to refer both to inhibitors of phosphate transporter activity and/or to phosphono-carboxylic acids (or pharmaceutically acceptable derivatives thereof).

The active agent will normally be administered to a patient in association with a pharmaceutically acceptable carrier although it will be appreciated that active agents may also be used without carrier material. Suitable carriers include solid, semi solid or liquid diluents, or ingestible capsules.

The medicaments in accordance with the invention may be formulated with reference to the epithelia damage of which they are intended to prevent and/or treat. For example, medicaments intended for the prevention and/or treatment of damage to “accessible” epithelia, such as the digestive epithelium of the mouth or the epithelium of the scalp, may be formulated for topical application.

In contrast, medicaments intended for the prevention and/or treatment of damage to “inaccessible” epithelia, such as the digestive epithelium of the small intestine or colon may be formulated for systemic administration (e.g. by oral or rectal or inhalation administration) such that it enters the blood stream and is then delivered to the epithelial target. Alternatively the active agent may be ingested and will then act directly on the digestive epithelia as it passes through the gastrointestinal tract.

Suitable formulations for topical administration include solutions, suspensions, jellies, gels, creams, ointments, sprays, foams, powders, liposomes, pastilles, chewing gums, toothpastes and mouth washes. In the case of topical application to digestive epithelia it may be particularly preferred to formulate the medicaments for oral administration, or for rectal administration (for example as suppositories), and in the case of topical application to the scalp the medicaments may be formulated as shampoos. In the case of non-viral damage to the respiratory epithelium the medicaments may be formulated as, for example, nasal drops, intranasal sprays or aerosols for inhalation.

Topical compositions suitable for application to the skin may include moisturisers, and sun tan lotions and creams. Such compositions are particularly suitable for the administration of active agents for prevention and/or treatment of epithelial damage, such as sun burn and blistering, caused by solar radiation.

In the case of topically applied compositions to be applied to the skin the vehicle used to carry the active agent may need to be one capable of crossing the keratinous layer of the skin. Examples of suitable vehicles for this purpose include dimethyl sulphoxide and acetic acid. The amount of active agent provided by such topical compositions is subject to variation, but typically may be between 0.05%-20% active agent by weight. Many methods are known for preparation of compositions for topical application. For example, the active agent may be mixed with know carrier materials such as isopropanol, glycerol, paraffin, stearyl alcohol, polyethylene glycol, and the like.

Suitable compositions may also include a known chemical absorption promoter. Examples of absorption promoters are e.g. dimethylacetamide (U.S. Pat. No. 3,472,931), trichloroethanol or trifluoroethanol (U.S. Pat. No. 3,891,757) certain alcohols and mixtures thereof (British Pat. No. 1,001,949). A carrier material for topical application to unbroken skin is also described in the British patent specification No. 1,464,975 which discloses a carrier material consisting of a solvent comprising 40-70% (v/v) isopropanol and 0-60% (v/v) glycerol, the balance, if any, being an inert constituent of a diluent not exceeding 40% of the total volume of solvent.

Alternatively, the skilled person will appreciate that topical administration may be achieved by means of localised injection, for example intra-dermal injection.

The medicaments may advantageously comprise the active agent formulated for systemic administration. For example the active agent may be formulated in a form suitable for oral administration, such as a tablet, effervescent powder, capsule, dragee or liquid preparation. Alternatively, systemic administration may be achieved by other routes, such as rectal administration (in which case the active agent may be formulated as a suppository) and nasal administration (by means of, for example, nasal sprays or aerosols suitable for inhalation). Suitable formulations for systemic administration also include injectable formulations, wherein the active agent may, for example, comprise an aqueous solution of a water soluble pharmaceutically acceptable salt of phosphono-carboxylic acid. Injectable formulations may optionally include a stabilising agent and/or buffer substances in aqueous solution, for instance a neutral buffered saline solution. Injectable formulations may contain the active agent in a concentration of 0.5-10%. Dosage units of the solution may be advantageously be provided in the form of ampoules.

In preparing medicaments of the invention suitable for oral administration, the active agent may be mixed with a solid, pulverulent carrier in order to form tablets, dragees and the like. Such carriers may be compressed to form tablets or cores of dragees. Examples of suitable carriers include lactose, saccharose, sorbitol and mannitol, starches such as potato starch, amylopectin, laminaria powder or citrus pulp powder, cellulose derivatives or gelatine. and also may include lubricants such as magnesium or calcium stearate or a Carbowax® or other polyethylene glycol waxes. If dragees are required, the cores may be coated, for example with concentrated sugar solutions which may contain gum arabic, talc and/or titanium dioxide, or alternatively with a film forming agent dissolved in easily volatile organic solvents or mixtures of organic solvents. Dyestuffs can be added to these coatings, for example, to distinguish between different contents of active agent. For the preparation of soft gelatine capsules consisting of gelatine and, for example, glycerol as a plasticizer, or similar closed capsule, the active agent may be admixed with a Carbowax® or a suitable oil such as sesame oil, Olive oil, or arachis oil. Hard gelatine capsules may contain granulates of the active agent with solid, pulverulent carriers such as lactose, saccharose, sorbitol, mannitol, starches (for example potato starch, corn starch or amylopectin), cellulose derivatives or gelatine, and may also include magnesium stearate or stearic acid as lubricants.

Medicaments in accordance with the present invention which are to be used in the treatment and/or prevention of non-viral damage to digestive epithelia may be formulated as tablets, capsules, or the like, for oral administration. It will be appreciated that when administered in this fashion the medicaments may be subject to degradation within the gastrointestinal tract, which may reduce the effectiveness of the active agent, and hence of the medicament. It will further be appreciated that it may be desired to treat non-viral damage occurring at one or more specific site(s) in the gastrointestinal tract, rather than treating the gastrointestinal tract as a whole. It may therefore be preferred to provide medicaments for oral administration with coatings that confer, at least partial, resistance to digestion. Such coatings may also be used to provide formulations giving sustained or delayed release of the active agent. Many methods are known for producing such coatings.

For example, sustained release tablets may be produced by using several layers of an active agent, separated by slowly dissolving coatings. Another way of preparing sustained release tablets is to divide the dose of the active agent into granules which are provided with coatings of different thicknesses. Such granules may be administered as the contents of capsules, or the granules, together with a carrier substance, may be compressed to form tablets. The active agent may also be incorporated in slowly dissolving tablets made for instance of fat and wax substances such as a physiologically inert plastic substance.

Similar coatings may be used for the production of medicaments formulated to release the active agent at a specific site. For example, tablets, etc. may be provided with an “enteric” coating, that is to say provided with a layer of a gastric juice-resistant enteric film or coating having such properties that it is not dissolved at the acidic pH found in the stomach. In such an enteric-coated medicament the active agent will not be released until the preparation reaches the intestines. Many example of suitable enteric coatings are known, and include cellulose acetate phtalate and hydroxypropulmethylcellulose phtalates (such as those sold under trade names HP 55 and HP 50, and Eudragit®L and Eudragit®S).

Effervescent powders provide a further preferred embodiment in which medicaments according to the invention may be formulated for oral administration. Such powders may be prepared by mixing the active agent with non-toxic carbonates or hydrogen carbonates (such as calcium carbonate, potassium carbonate and potassium hydrogen carbonate), and/or with solid, non-toxic acids (such as tartaric acid, ascorbic acid, and citric acid). Effervescent powders may also be provided with suitable flavourings and/or sweeteners to improve palatability.

Liquid preparations for oral application represent a further form in which medicaments according to the invention may be formulated for oral administration. Suitable forms of liquid preparations include elixirs, syrups or suspensions. Such liquid preparations may comprise from about 0.1% to 20% by weight of active agent, and may further comprise ingredients such as sugar, ethanol, water, glycerol, propylene glycol, and flavourings and/or sweeteners. Liquid preparations may also include a dispersing agent, such as carboxymethylcellulose.

The dosage at which the active agents are administered may be varied in response to a number of factors. For example, in the case of epithelial damage caused by non-viral microbial infection, the amount of the active agent to be administered may be influenced by the severity of the infection. The amount of active agent required may also vary depending on factors such as the age of the patient being treated and the area of epithelium damaged.

It will be appreciated that pharmaceutical compositions containing active agents may be suitably formulated so that they provide doses within the ranges contemplated herein, either as a single dose or in the form of multiple dosage units.

Generally when medicaments of the invention are used to treat existing epithelial damage, or conditions caused or characterised thereby, the medicaments should be administered as soon as the damage has occurred or the condition has been diagnosed. However, such damage or conditions can develop over days or even weeks. Therefore the subject being treated may well benefit by administration of a medicament of the invention, even if it is administered days or even weeks after the damage occurred or the condition was developed or diagnosed. Therapeutic use of the medicament may continue until the damage or condition has resolved to a clinician's satisfaction.

When used as a prophylactic (e.g. before beginning cancer therapy such as chemotherapy or radiotherapy) the medicaments of the invention should be administered as soon as the risk of epithelial damage has been recognised. For instance, it may be preferred to administer the medicament at the time of treatment with the cancer therapy, or in the hours or days preceding the treatment.

Medicaments manufactured according to the invention may be formulated such that they provide a daily dose of up to 500 mg of the active agent per kilogram bodyweight to a person receiving the medicament. Preferably the medicaments may be formulated such that they provide a daily dose of up to 250 mg per kilogram bodyweight, more preferably up to 120 mg per kilogram bodyweight, even more preferably up to 60 mg per kilogram bodyweight. Medicaments in accordance with the invention may, for instance provide a daily dose of 50 mg per kilogram bodyweight, or more preferably still 30 mg, 15 mg or 5 mg per kilogram, and most preferably 1 mg per kilogram.

The preferred frequency of administration will depend upon the biological half-life of the selected active agent. Typically a medicament in accordance with the invention should be administered to a target tissue such that the concentration of the active agent in the epithelium damaged, or at risk of damage, is maintained at a level suitable to achieve a therapeutic effect. This may require administration daily or even several times daily.

The inventors have found that in a preferred embodiment of the invention an active agent in accordance with the invention may be administered before administration of an agent causing non-viral epithelial damage (for example, before administration of a chemotherapeutic agent, or radiotherapy). For example the active agent may be administered up to 24 hours before the onset of epithelial damage, more preferably up to twelve hours before onset of damage, and most preferably an hour before damage.

Administration of the active agent may be repeated during the period following the administration of the damaging agent. Thus, for example, the active agent may be administered on the first and subsequent days following administration of chemotherapy or radiotherapy, preferably for at least the first two days following the onset of damage, more preferably for at least the three days following onset of damage, and most preferably for at least the seven days after damage.

It is particularly preferred that an active agent in accordance with the invention may be administered both before and after the onset of damage. By way of example, the inventors have found that medicaments of the invention are particularly effective if administered both prior to the onset of epithelial damage and for at least the three days following damage.

The inventors have surprisingly found that when multiple doses of an agent capable of causing epithelial damage, such as a chemotherapy agent or radiation, are to be administered medicaments comprising active agents may be particularly effective if administered in a single dose before the onset of damage, rather than before or after each administration of the damaging agent.

It will be appreciated that, while the preceding paragraphs provide non-limiting examples of possible and preferred regimes for the administration of active agents, known procedures, such as those conventionally employed by the pharmaceutical industry (e.g. in vivo experimentation, clinical trials etc), may be used to establish specific formulations of compositions and precise therapeutic regimes (such as daily doses of the active agent and the frequency of administration).

According to an ninth aspect of the invention there is provided a shampoo composition comprising an inhibitor of phosphate transporter activity and at least one surface active agent suitable for shampooing hair.

In a tenth aspect of the invention there is provided a shampoo composition comprising a phosphono-carboxylic acid, or pharmaceutically acceptable derivative thereof, and at least one surface active agent suitable for shampooing hair.

A “shampoo” as considered in the present invention may be taken to comprise any product used in the cleaning, conditioning, styling or maintenance of hair. For example, a shampoo according to the invention may be a medicated shampoo, a shampoo having anti-dandruff properties, a conditioner, a shower gel or body wash. Active agents in accordance with the invention may also be provided by means of hair gels, waxes, creams or other styling preparations.

The shampoo composition may preferably comprise the active agent alpha-C₁-alpha-Br-phosphonoacetate, however it will be appreciated that the range of active agents considered for use in medicaments prepared in accordance with the invention are also suitable for use in shampoos in accordance with the invention.

The ability of inhibitors of phosphate transporter activity and/or phosphono-carboxylic acids (or pharmaceutically acceptable derivatives thereof) to prevent and/or treat epithelial damage arising as a result of chemotherapy is of particularly notable value. Accordingly it is preferred that medicaments prepared in accordance with the first or second aspects of the invention are formulated to produce a medicament for use in combination with a chemotherapeutic compound.

Indeed, according to a eleventh aspect of the invention, there is provided the combination of an inhibitor of phosphate transporter activity and a chemotherapeutic compound. Furthermore, according to a twelfth aspect of the invention there is provided the combination of a phosphon-carboxylic acid, or pharmaceutically acceptable derivative thereof, and a chemotherapeutic compound. The inhibitor of phosphate transporter activity or the phosphono-carboxylic acid (or pharmaceutically acceptable derivative thereof) may be selected and formulated as described for medicaments according to the invention.

Combinations in accordance with the eleventh and twelfth aspects of the invention may be used to prevent epithelial damage occurring in patients undergoing chemotherapy, or, in the case of chemotherapy patients already suffering from epithelial damage or a condition characterised by such damage, to allow chemotherapy to continue while preventing further damage occurring. It will be appreciated that such combinations are particularly suitable for use in contexts in which a chemotherapeutic drug is administered to treat cancers of the gastrointestinal tract.

A preferred active agent suitable for use in combinations of the eleventh or twelfth aspects of the invention is alpha-Cl-alpha-Br-phosphonoacetate. The chemotherapeutic compound may preferably be fluorouracil, which is the chemotherapeutic drug most commonly used in cancers of the gastrointestinal tract. Other chemotherapeutic compounds that may advantageously be utilised in combinations according to the invention include doxyrubicin (Adriamycin) daunorubicin, methotrexate, vincristine, vinblastine, Melphalan, cytosine arabinoside, thioguanine, bleomycin, dactinomycin, cisplatin, mithramycin, hydroxyurea and procarbazine hydrochloride, all of which are known to cause mucositis.

Combinations according to the present invention may be combinations in which the active agent and chemotherapeutic compound are provided in separate dosage forms. Alternatively combinations may comprise the admixture of the active agent and chemotherapeutic compound in dosage form.

By “dosage form” is meant a form suitable for administration, and comprising a dose of the selected active agent and/or the chemotherapeutic compound. Such dosages may be determined according to the amount of the active agent or chemotherapeutic compound required, and the frequency of administration desired. Thus a dosage form may, for example, comprise a weekly dose of the active agent and chemotherapeutic compound to be administered, or a daily dose, or a fraction of a daily dose.

Experimental data will now be described with reference to the accompanying drawings, in which;

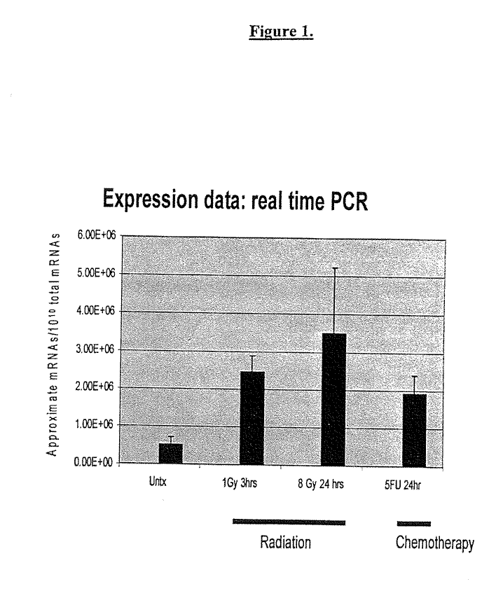

FIG. 1 illustrates the increase in expression of phosphate transporters in response to epithelial damage caused by radiation or cytotoxic chemicals;

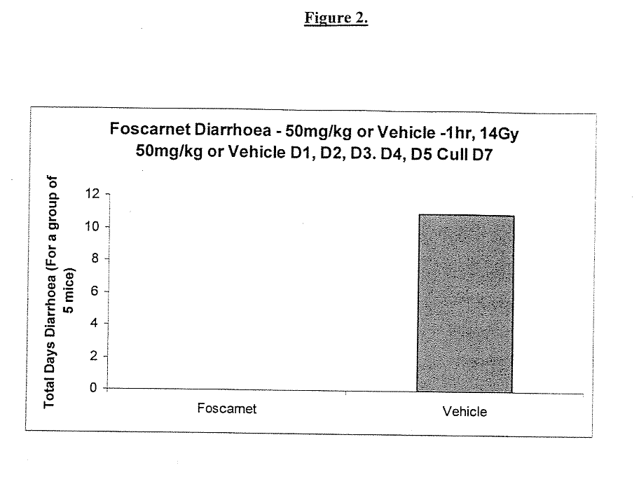

FIG. 2 illustrates the incidences of diarrhoea in irradiated mice treated either with foscarnet, or with vehicle control;

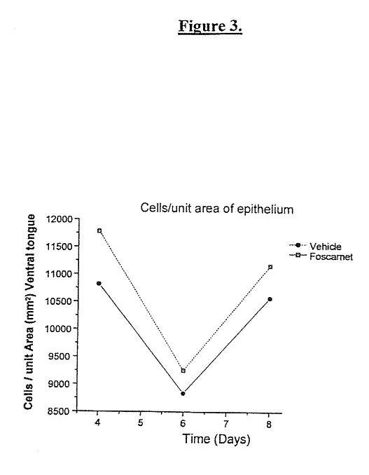

FIG. 3 illustrates the overall cellularity of the epithelium covering the ventral surface of the tongue over time in control-treated or foscarnet-treated animals given the chemotherapy agent 5FU;

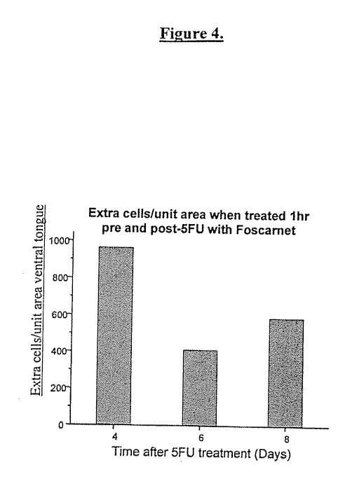

FIG. 4 compares numbers of cells per unit area in the epithelium covering the ventral surface of the tongue in foscarnet treated and control treated animals;

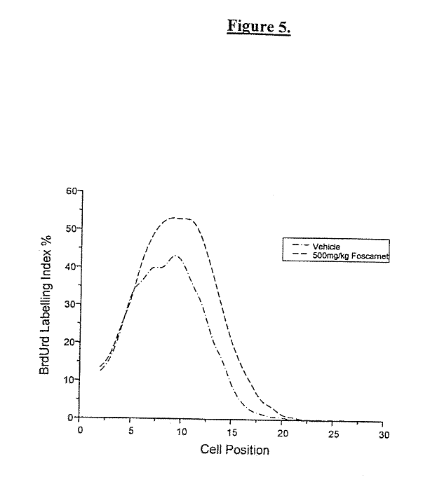

FIG. 5 illustrates bromodeoxyuridine labelling index in intestinal crypt cells of foscarnet-treated and control-treated animals;

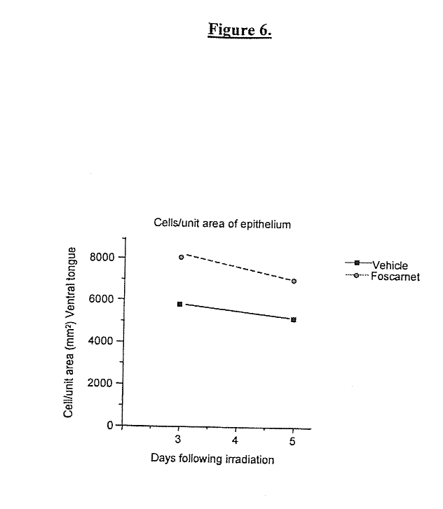

FIG. 6 compares the change with time in the number of cells per unit area on the ventral surfaces of the tongues of foscarnet-treated and vehicle-treated animals; and

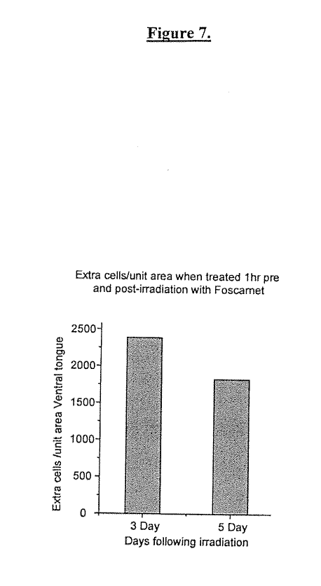

FIG. 7 compares the average number of extra cells present per unit area in the epithelium covering the ventral surface of the tongues of foscarnet-treated and vehicle-treated animals.

### EXPERIMENTAL DATA

### Example 1

### Investigation of Regulation of Phosphate Transporter Expression in Response to Epithelial Damage

Mice were subjected to two different experimental models of epithelial damage, one causing chemical damage and the other radiation damage, as set out below:

### Radiation Damage Models:

A first group of mice (n=6) were treated with 1 Gy X-ray whole body radiation at a does of 0.7 Gy/minute and killed 3 hours after radiation treatment and tissues of the gastrointestinal tract collected for investigation.

A second group of mice (n=6) were treated with 8 Gy X-ray whole body radiation at a dose rate of 0.7 Gy/minute. Treated mice were killed 24 hours after radiation treatment, and tissues of the gastrointestinal tract collected for investigation.

### Chemical Damage Model:

A third group of mice (n=6) were given two IP injections of 5-fluorouracil at 40 mg/kg body weight 6 hours apart. Treated mice were killed 24 hours after the second 5-fluorouracil treatment, and tissues of the gastrointestinal tract collected for investigation.

The treatments used further provide models of therapies administered to patients undergoing treatment for cancer. The radiation damage models provide models of radiotherapy, and the chemical damage model provides a model of chemotherapy.

Total RNA was prepared from each sample using an RNAqueous™ 96 RNA isolation kit (Ambion). The effect of epithelial damage on phosphate transporter expression was investigated by quantitative real-time PCR analysis of representative total cDNA (Al Taher, A., Bashein, A., Nolan, T., Hollingsworth, M., and Brady, G. (2000). Global cDNA amplification combined with real-time RT-PCR: accurate quantification of multiple human potassium channel genes at the single cell level. Comp Funct Genomics: Yeast 17, 201-210. Brady, G., Barbara, M., and Iscove, N. N. (1990). Representative in vitro cDNA amplification from individual hemopoietic cells and colonies. Meth Mol Cell Biol 2, 17-25).

Real-time RT-PCR data was obtained using the Eurogentec SYBR Green™ core kit as outlined in the manufacturer's instructions, and performed on the ABI Prism™ 7000 Sequence Detection system. The oligonucleotides used for quantitative analysis of Pit1, also known as the Mus musculus solute carrier family 20, member 1 (Slc20a1), were:

5′-GCGGTTGTGGTTATTCTTCTGAG-3′ (sense)
and
5′ CCCAAAGTTCACATTCCACTTCA-3′ (anti-sense).

These oligonucleotides were based on sequence accession number—NM_{—}015747.1. FIG. 1 shows data for Pit1 expression in colon tissue collected from radiation treated, chemical treated and control animals. For each group the data presented is the average of six independent animals, error bars indicate standard deviation.

The radiation treatment labelled 1 Gy 3 hours mice corresponds to the first experimental group of animals (treated with 1 Gy X-ray whole body radiation at a dose rate of 0.7 Gy/minute, killed 3 hours after radiation treatment), whilst the radiation treatment labelled 8 Gy 24 hours mice corresponds to the second experimental group of animals (treated with 8 Gy X-ray whole body radiation at a dose rate of 0.7 Gy/minute and were killed 24 hours after radiation treatment). The treatment labelled 5FU 24 hrs mice corresponds to the third experimental group (receiving two IP injections of 5-fluorouracil at 40 mg/kg body weight 6 hours apart and killed 24 hours after the second 5-fluorouracil treatment).

In summary, the results shown in FIG. 1 illustrate that mRNA for Pit1 was significantly increased in the damaged digestive tissues of treated animals as compared to control untreated animals. These results confirm that a significant increase in Pit1 phosphate transporter mRNA occurs in response to both radiation-induced and chemical-induced damage, and furthermore that the response to radiation treatment was dose-dependent.

### Example 2

### Diarrhoea Prevention Assay

The ability of foscarnet (phosphonoformic acid trisodium salt hexahydrate) to prevent diarrhoea following radiological injury to digestive epithelia was investigated.

The model of radiological insult used was treatment with 14 Gy X-ray partial body radiation (head and thorax lead shielded) at a dose rate of 0.7 Gy/minute. Such radiation treatment provides a model of radiotherapy administered to patients undergoing treatment for cancer.

Three experimental groups, each of five mice, were established as set out below:

Group 1: Received intra-peritoneal injection, containing 50 mg foscarnet/kilogram bodyweight, in sterile water, one hour prior to radiation treatment. Received one further identical injection on each of the next five days following radiation treatment.
Group 2: Received intra-peritoneal injection, containing 100 mg foscarnet/kilogram bodyweight, in sterile water, one hour prior to radiation treatment. Received one further identical injection on each of the next five days following radiation treatment.
Group 3: Vehicle control. Group 1: Received intra-peritoneal injection, of sterile water, one hour prior to radiation treatment. Received one further identical injection on each of the next five days following radiation treatment.

The incidences of both diarrhoea (measured by assessing perianal soilage twice daily) and morbidity among experimental animals were measured over the course of the seven days following radiation treatment. The results are shown in Table 1 & FIG. 2 below:

TABLE 1

| | Day 3 | Day 4 | Day 5 | Day 6 | Day 7 |
|---|---|---|---|---|---|
| Control | No | 3 mice | All mice | 3 mice | All mice |
| | diarrhoea | had | had | had | moribund |
| | | diarrhoea | diarrhoea | diarrhoea | |
| 50 mg/kg | No | No | No | No | No diarrhoea |
| | diarrhoea | diarrhoea | diarrhoea | diarrhoea | |
| 100 mg/kg | No | No | No | 2 mice | 1 mouse |
| | diarrhoea | diarrhoea | diarrhoea | had | had |
| | | | | diarrhoea | diarrhoea |

As can be seen treatment with foscarnet at 50 mg per kilogram bodyweight protects completely against the deleterious effects of radiation treatment. Similarly, treatment with foscarnet at 100 mg per kilogram bodyweight results in reduced incidence of diarrhoea or associated morbidity compared to vehicle controls. These results illustrate the ability of the treatment of the invention to prevent diarrhoea, and associated morbidity, resulting from radiation damage to digestive epithelia.

### Example 3

### In Vivo Protection of Intestinal Epithelium Clonogenic Stem Cells

### Radiation Damage Independent Experiment 1

The ability of foscarnet (phosphonoformic acid trisodium salt hexahydrate) to prevent radiological damage to clonogenic stem cells of the digestive epithelium was illustrated by the following experiment.

The model of radiological insult used was treatment with 13 Gy X-ray whole body radiation at a dose rate of 0.7 Gy/minute. Such radiation treatment provides a model of radiotherapy administered to patients undergoing treatment for cancer.

Four experimental groups, each of six mice, were established as set out below:

Group 1: Received intra-peritoneal injection, containing 50 mg foscarnet/kilogram bodyweight, in sterile water, one hour prior to radiation treatment. Received one further identical injection on each of the three days following radiation treatment.
Group 2: Received intra-peritoneal injection, containing 100 mg foscarnet/kilogram bodyweight, in sterile water, one hour prior to radiation treatment. Received one further identical injection on each of the three days following radiation treatment.
Group 3: Vehicle control. Received intra-peritoneal injection, of sterile water, one hour prior to radiation treatment. Received one further identical injection on each of the three days following radiation treatment.
Group 4: Untreated controls. Received neither injections nor exposure to radiation.

On the fourth day after treatment the animals of each group were killed, and intestinal tissue harvested for histological analysis. Tissue sections were stained with haemotoxylin and eosin, and were analysed for the presence of regenerating intestinal crypts. Regenerating intestinal crypts are derived from one or more surviving clonogenic stem cells, whereas sterilised crypts (containing no surviving stem cells) disappear within two days of irradiation. The number of regenerating crypts per intestinal circumference was counted, and the mean crypt width measured. Scores for each animal were then corrected to account for the probability of preferentially scoring larger crypts according to the following equation:

$$Corrected number = \frac{average width of untreated crypts}{average width of treated crypts} \times number of crypts$$

$$For each treatment a Protection Factor was calculated using the following equation \text{:} \frac{corrected crypts / circumference treated}{corrected crypts / circumference untreated ( vehicle )}$$

The results are shown in Tables 2 and 3 below, and illustrate that the treatments administered to both Groups 1 and 2 (50 mg foscarnet and 100 mg foscarnet respectively) resulted in increased numbers of intestinal crypts in the experimental animals after exposure to radiation. This indicates that the treatments protected intestinal epithelium clonogenic stem cells since they are, by definition, the only cells capable of initiating such regeneration. The intestinal epithelium of mice treated with 50 mg foscarnet contained nearly 50% more crypts after 50 mg/kg foscarnet treatment (compared to that of mice treated with vehicle alone), indicating that at least 50% more small intestinal clonogenic stem cells survived.

Since each clonogenic stem cell is able produce an exponential number of daughter cells this greater than 50% increase in survival will, after cell expansion, cause a large increase in epithelial cellularity. Such cell survival and expansion can reduce diarrhoea, as illustrated in Example 1, as well as ulceration (mucositis) and other related conditions.

TABLE 2

| | | | crypt | corrected |
|---|---|---|---|---|
| Treatment | | no. crypts/ | width/ | crypts/ |
| 13Gy plus: | mouse no. | circumference | um | circumference |
| 50 mg foscarnet - | 1 | 10.7 | 50.7 | 6.1 |
| 1 hr, 1, 2, 3 day | 2 | 6.6 | 52.1 | 3.6 |
| | 3 | 7.2 | 48.0 | 4.3 |
| | 4 | 4.5 | 51.4 | 2.5 |
| | 5 | 4.8 | 52.3 | 2.6 |
| | 6 | | | |
| | Average | 6.8 | 50.9 | 3.8 |
| 100 mg | 1 | 5.5 | 51.8 | 3.0 |
| foscarnet - | 2 | 6.4 | 51.6 | 3.6 |
| 1 hr, 1, 2, 3 day | 3 | 4.3 | 48.7 | 2.5 |
| | 4 | 3.8 | 51.4 | 2.1 |
| | 5 | 8.0 | 47.8 | 4.8 |
| | 6 | | | |
| | Average | 5.6 | 50.3 | 3.2 |
| vehicle | 1 | 5.1 | 53.4 | 2.7 |
| | 2 | 4.9 | 54.8 | 2.6 |
| | 3 | 6.6 | 48.9 | 3.9 |
| | 4 | 2.4 | 54.2 | 1.3 |
| | 5 | 4.3 | 54.8 | 2.3 |
| | 6 | 4.5 | 52.9 | 2.4 |
| | Average | 4.6 | 53.2 | 2.5 |
| untreated | 1 | 115.4 | 30.1 | 109.9 |
| control | 2 | 110.5 | 27.8 | 114.1 |
| | 3 | 110.2 | 28.3 | 111.7 |
| | 4 | 113.1 | 30.5 | 106.3 |
| | 5 | 108.4 | 27.7 | 112.5 |
| | 6 | 102.5 | 27.8 | 106.0 |
| | Average | 110.0 | 28.7 | 110.0 |

TABLE 3

| Treatment | Protection Factor |
|---|---|
| 50 mg foscarnet - 1 hr, 1, 2, 3 day | 1.52 |
| 100 mg foscarnet - 1 hr, 1, 2, 3 day | 1.28 |

### Example 4

### In Vivo Protection of Intestinal Epithelium Clonogenic Stem Cells

### Chemotherapy Drug Damage

The ability of foscarnet (phosphonoformic acid trisodium salt hexahydrate) to prevent cytotoxic damage to clonogenic stem cells of the digestive epithelium was illustrated by the following experiment.

The model of cytotoxic insult used was treatment with 2 doses of 5-Fluorouracil 6 hours apart at either 400 mg or 500 mg of 5-Fluorouracil/kilogram bodyweight. Such cytotoxic treatment provides a model of chemotherapy administered to patients undergoing treatment for cancer.

Five experimental groups, each of five mice, were established as set out below:

Group 1: Received intra-peritoneal injection, containing 50 mg foscarnet/kilogram bodyweight, in sterile water, one hour prior to the first cytotoxic treatment (400 mg 5-Fluorouracil/kilogram bodyweight). Received one further identical injection on each of the three days following cytotoxic treatment.
Group 2: Received intra-peritoneal injection, containing 50 mg foscarnet/kilogram bodyweight, in sterile water, one hour prior to both cytotoxic treatments (400 mg 5-Fluorouracil/kilogram bodyweight). Received one further identical injection on each of the three days following cytotoxic treatment.
Group 3: Vehicle control. Received intra-peritoneal injection, of sterile water, one hour prior to both cytotoxic treatments (400 mg 5-Fluorouracil/kilogram bodyweight). Received one further identical injection on each of the three days following cytotoxic treatment.
Group 4: Received intra-peritoneal injection, containing 50 mg foscarnet/kilogram bodyweight, in sterile water, one hour prior to both cytotoxic treatments (500 mg 5-Fluorouracil/kilogram bodyweight). Received one further identical injection on each of the three days following cytotoxic treatment.
Group 5: Vehicle control. Received intra-peritoneal injection, of sterile water, one hour prior to both cytotoxic treatments (500 mg 5-Fluorouracil/kilogram bodyweight). Received one further identical injection on each of the three days following cytotoxic treatment.

On the fourth day after treatment the animals of each group were killed, and intestinal tissue harvested for histological analysis. Tissue sections were stained with haemotoxylin and eosin, and were analysed for the presence of regenerating intestinal crypts. Regenerating intestinal crypts are derived from one or more surviving clonogenic stem cells, whereas sterilised crypts (containing no surviving stem cells) disappear within two days of irradiation. The number of regenerating crypts per intestinal circumference was counted, and the mean crypt width measured. Scores for each animal were then corrected to account for the probability of preferentially scoring larger crypts according to the following equation:

$$Corrected number = \frac{average width of untreated crypts}{average width of treated crypts} \times number of crypts$$

$$For each treatment a Protection Factor was calculated using the following equation \text{:} \frac{corrected crypts / circumference treated}{corrected crypts / circumference untreated ( vehicle )}$$

The results are shown in Tables 4 and 5 below, and illustrate that the treatments administered to both Groups 1, 2 and 4 resulted in increased numbers of intestinal crypts in the experimental animals after exposure to 5-Fluorouracil. This indicates that the treatments protected intestinal epithelium clonogenic stem cells since they are, by definition, the only cells capable of initiating such regeneration. The intestinal epithelium of mice from group 1 contained nearly 50% more crypts after 50 mg/kg foscarnet treatment (compared to that of mice treated with vehicle alone), indicating that at least 50% more small intestinal clonogenic stem cells survived.

Since each clonogenic stem cell is able to produce an exponential number of daughter cells this greater than 50% increase in survival will, after cell expansion, cause a large increase in epithelial cellularity. Such cell survival and expansion can reduce diarrhoeas well as ulceration (mucositis) and other related conditions.

TABLE 4

| | | | | corrected |
|---|---|---|---|---|
| | | no. crypts/ | crypt | crypts/ |
| | | circum- | width/ | circum- |
| Treatment: | mouse no. | ference | um | ference |
| 50 mg foscarnet - | 1 | 12.4 | 32.47 | 11.0 |
| 1 hr prior to 1^{st} | 2 | 24.1 | 29.79 | 23.2 |
| dose of 5-Flurouracil | 3 | 17.9 | 29.81 | 17.2 |
| (400 mg/kg x2 6 hrs apart) | 4 | 17.9 | 31.12 | 16.5 |
| and D1, D2, D3 | 5 | 35.2 | 31.44 | 32.1 |
| | Average | 21.5 | 30.93 | 20.0 |
| 50 mg foscarnet - | 1 | 15.9 | 31.84 | 14.3 |
| 1 hr prior to both | 2 | 16.8 | 29.66 | 16.3 |
| doses of 5-Flurouracil | 3 | 22.8 | 31.83 | 20.6 |
| (400 mg/kg x2 6 hrs apart) | 4 | 11.4 | 28.80 | 11.4 |
| and D1, D2, D3 | 5 | 23.4 | 29.84 | 22.5 |
| | Average | 18.1 | 30.39 | 17.0 |
| vehicle | 1 | 9.3 | 26.91 | 9.9 |
| 1 hr prior to both | 2 | 13.9 | 27.87 | 14.3 |
| doses of 5-Flurouracil | 3 | 12.8 | 29.97 | 12.3 |
| (400 mg/kg x2 6 hrs apart) | 4 | 12.9 | 30.27 | 12.2 |
| and D1, D2, D3 | 5 | 4.8 | 29.86 | 4.6 |
| | Average | 10.7 | 28.98 | 10.7 |
| 50 mg foscarnet | 1 | 8.5 | 28.66 | 8.5 |
| 1 hr prior to both | 2 | 13.0 | 28.59 | 13.0 |
| doses of 5-Flurouracil | 3 | 17.9 | 31.53 | 16.3 |
| (500 mg/kg x2 6 hrs apart) | 4 | 6.0 | 30.86 | 5.6 |
| and D1, D2, D3 | 5 | 7.3 | 27.67 | 7.6 |
| | Average | 10.5 | 29.46 | 10.2 |
| Vehicle | 1 | 3.3 | 29.17 | 3.2 |
| 1 hr prior to both | 2 | 0.8 | 26.14 | 0.9 |
| doses of 5-Flurouracil | 3 | 7.9 | 26.72 | 8.5 |
| (500 mg/kg x2 6 hrs apart) | 4 | 8.3 | 25.33 | 9.4 |
| and D1, D2, D3 | 5 | 12.0 | 26.17 | 13.2 |
| | Average | 6.5 | 26.71 | 7.0 |

TABLE 5

| Treatment | Protection Factor |
|---|---|
| 50 mg foscarnet 1 hr prior to 1^{st} dose of 5- | 1.87 |
| Flurouracil (400 mg/kg x2 6 hrs apart) and D1, D2 | |
| & D3 | |
| 50 mg foscarnet 1 hr prior to both doses of 5- | 1.59 |
| Flurouracil (400 mg/kg x2 6 hrs apart) and D1, D2 | |
| & D3 | |
| 50 mg foscarnet 1 hr prior to both doses of 5- | 1.46 |
| Flurouracil (500 mg/kg x2 6 hrs apart) and D1, D2 | |
| & D3 | |

### Example 5

### In Vivo Protection of Intestinal Epithelium Clonogenic Stem Cells

### Radiation Damage Independent Experiment 2

The ability of foscarnet (phosphonoformic acid trisodium salt hexahydrate) to prevent radiological damage to clonogenic stem cells of the digestive epithelium was illustrated by the following experiment.

The model of radiological insult used was treatment with 13 Gy X-ray whole body radiation at a dose rate of 0.7 Gy/minute. Such radiation treatment provides a model of radiotherapy administered to patients undergoing treatment for cancer.

Three experimental groups, each of six mice, were established as set out below:

Group 1: Received intra-peritoneal injection, containing 50 mg foscarnet/kilogram bodyweight, in sterile water, one hour prior to radiation treatment. Received one further identical injection on each of the three days following radiation treatment.
Group 2: Received intra-peritoneal injection, containing 25 mg foscarnet/kilogram bodyweight, in sterile water, one hour prior to radiation treatment. Received one further identical injection on each of the three days following radiation treatment.
Group 3: Vehicle control. Received intra-peritoneal injection, of sterile water, one hour prior to radiation treatment. Received one further identical injection on each of the three days following radiation treatment.

On the fourth day after treatment the animals of each group were killed, and intestinal tissue harvested for histological analysis. Tissue sections were stained with haemotoxylin and eosin, and were analysed for the presence of regenerating intestinal crypts. Regenerating intestinal crypts are derived from one or more surviving clonogenic stem cells, whereas sterilised crypts (containing no surviving stem cells) disappear within two days of irradiation. The number of regenerating crypts per intestinal circumference was counted, and the mean crypt width measured. Scores for each animal were then corrected to account for the probability of preferentially scoring larger crypts according to the following equation;

$$Corrected number = \frac{average width of untreated crypts}{average width of treated crypts} \times number of crypts$$

$$For each treatment a Protection Factor was calculated using the following equation \text{:} \frac{corrected crypts / circumference treated}{corrected crypts / circumference untreated ( vehicle )}$$

The results are shown in Tables 6 and 7 below, and illustrate that the treatments administered to both Groups 1 and 2 (50 mg foscarnet and 25 mg foscarnet respectively) resulted in increased numbers of intestinal crypts in the experimental animals after exposure to radiation. This indicates that the treatments protected intestinal epithelium clonogenic stem cells since they are, by definition, the only cells capable of initiating such regeneration. The intestinal epithelium of mice treated with 50 mg foscarnet contained nearly 50% more crypts after 50 mg/kg foscarnet treatment (compared to that of mice treated with vehicle alone), indicating that at least 50% more small intestinal clonogenic stem cells survived.

Since each clonogenic stem cell is able produce an exponential number of daughter cells this greater than 50% increase in survival will, after cell expansion, cause a large increase in epithelial cellularity. Such cell survival and expansion can reduce diarrhoea, as well as ulceration (mucositis) and other related conditions.

TABLE 6

| | | | crypt | corrected |
|---|---|---|---|---|
| Treatment | | no. crypts/ | width/ | crypts/ |
| 13Gy plus: | mouse no. | circumference | um | circumference |
| 50 mg foscarnet - | 1 | 5.2 | 55.6 | 2.7 |
| 1 hr, 1, 2, 3 day | 2 | 4.1 | 61.9 | 1.9 |
| | 3 | 9.6 | 51.1 | 5.4 |
| | 4 | 13.2 | 50.5 | 7.5 |
| | 5 | 11.8 | 52.0 | 6.5 |
| | 6 | 8.7 | 57.1 | 4.4 |
| | Average | 8.8 | 54.7 | 4.7 |
| 25 mg foscarnet - | 1 | 2.6 | 52.5 | 1.4 |
| 1 hr, 1, 2, 3 day | 2 | 4.1 | 59.2 | 2.0 |
| | 3 | 5.4 | 57.4 | 2.7 |
| | 4 | 9.4 | 60.7 | 4.4 |
| | 5 | 6.3 | 59.5 | 3.0 |
| | 6 | 6.4 | 55.1 | 3.3 |
| | Average | 5.7 | 57.4 | 2.8 |
| vehicle | 1 | 2.5 | 62.5 | 1.1 |
| | 2 | 4.4 | 50.9 | 2.5 |
| | 3 | 5.3 | 58.4 | 2.6 |
| | 4 | 1.6 | 49.1 | 0.9 |
| | 5 | 5.9 | 64.6 | 2.6 |
| | 6 | 10.7 | 55.3 | 5.6 |
| | Average | 5.0 | 56.8 | 2.5 |

TABLE 7

| Treatment | Protection Factor |
|---|---|
| 50 mg foscarnet - 1 hr, 1, 2, 3 day | 1.88 |
| 25 mg foscarnet - 1 hr, 1, 2, 3 day | 1.12 |

### Example 6

### In Vivo Protection of Oral Mucosa Following Chemotherapy Damage

The ability of foscarnet (phosphonoformic acid trisodium salt hexahydrate) to prevent cytotoxic damage to oral mucosa was illustrated by the following experiment.

The model of cytotoxic insult used was treatment with 2 doses of 400 mg/kilogram bodyweight 5-Fluorouracil 6 hours apart. Such cytotoxic treatment provides a model of chemotherapy administered to patients undergoing treatment for cancer.

Experimental groups, each of five mice, were established as set out below:

Group 1: Untreated control.
Group 2: Received intra-peritoneal injection, containing 50 mg foscarnet/kilogram bodyweight, in sterile water, one hour prior to the first cytotoxic treatment (400 mg 5-Fluorouracil/kilogram bodyweight). Received one further identical injection of foscarnet (50 mg/kilogram bodyweight) on each of the three days following cytotoxic treatment. On the fourth day after treatment the animals were killed, and oral tissue harvested for analysis.
Group 3: Received intra-peritoneal injection, containing 50 mg foscarnet/kilogram bodyweight, in sterile water, one hour prior to the first cytotoxic treatment (400 mg 5-Fluorouracil/kilogram bodyweight). Received one further identical injection of foscarnet (50 mg/kilogram bodyweight) on each of the three days following cytotoxic treatment. On the sixth day after treatment the animals were killed, and oral tissue harvested for analysis.
Group 4: Received intra-peritoneal injection, containing 50 mg foscarnet/kilogram bodyweight, in sterile water, one hour prior to the first cytotoxic treatment (400 mg 5-Fluorouracil/kilogram bodyweight). Received one further identical injection of foscarnet (50 mg/kilogram bodyweight) on each of the three days following cytotoxic treatment. On the eighth day after treatment the animals were killed, and oral tissue harvested for analysis.
Group 5: Vehicle control. Received intra-peritoneal injection, of sterile water, one hour prior to both cytotoxic treatments (400 mg 5-Fluorouracil/kilogram bodyweight). Received one further identical injection of water on each of the three days following cytotoxic treatment. On the fourth day after treatment the animals were killed, and oral tissue harvested for analysis.
Group 6: Vehicle control. Received intra-peritoneal injection, of sterile water, one hour prior to both cytotoxic treatments (400 mg 5-Fluorouracil/kilogram bodyweight). Received one further identical injection of water on each of the three days following cytotoxic treatment. On the sixth day after treatment the animals were killed, and oral tissue harvested for analysis.
Group 7: Vehicle control. Received intra-peritoneal injection, of sterile water, one hour prior to both cytotoxic treatments (400 mg 5-Fluorouracil/kilogram bodyweight). Received one further identical injection of water on each of the three days following cytotoxic treatment. On the eighth day after treatment the animals were killed, and oral tissue harvested for analysis.

Tissue sections were stained with thionin and using a Zeiss AxioHOME the number of cells were assessed in both the basal and suprabasal layers of the ventral surface of the tongue. The area from the basal layer to the stratum corneum stratum granulosum interface was measured along with the length of the basal layer. This was performed in 5 consecutive areas 2 mm back from the tip of the tongue. From these measurements the damage that the 5FU had caused to the tongue could be assessed as overall cellularity of the tongue or the total number of cells/unit area (mm²).

The results of Example 6 are shown in FIGS. 3 and 4.

Foscarnet (50 mg/kg bodyweight) or vehicle alone was administered one hour prior to two injections of 5FU occurring six hours apart from one another. Foscarnet or vehicle were then further administered daily for three days.

Time referred to in FIGS. 3 and 4 is the number of days following 5FU treatment.

FIG. 3 illustrates the overall cellularity of the epithelium covering the ventral surface of the tongue over time after administration of the chemotherapy agent 5FU. Lines show values for both foscarnet treatment and vehicle alone.

FIG. 4 compares numbers of cells per unit area in the epithelium covering the ventral surface of the tongue in foscarnet treated and control treated animals. The results show the number of extra cells per unit area gained by foscarnet treatment.

### Example 7

### In Vivo Protection of Intestinal Epithelium Clonogenic Stem Cells

### Chemotherapy Drug Damage

### Independent Experiment 2

The data presented in Example 4 were expanded in the following study, in which the ability of foscarnet (phosphonoformic acid trisodium salt hexahydrate) to prevent cytotoxic damage to clonogenic stem cells of the digestive epithelium was investigated using the following experiment.

The model of cytotoxic insult used was treatment with 2 doses of 5-Fluorouracil 6 hours apart at 400 mg of 5-Fluorouracil/kilogram bodyweight. Such cytotoxic treatment provides a model of chemotherapy administered to patients undergoing treatment for cancer.

8 experimental groups, each of six mice, were established as set out below:

Group 1: Received intra-peritoneal injection, containing 50 mg foscarnet/kilogram bodyweight, in sterile water, one hour prior to the first cytotoxic treatment. Received one further identical injection on each of the three days following cytotoxic treatment.
Group 2: Received intra-peritoneal injection, containing 50 mg foscarnet/kilogram bodyweight, in sterile water, one hour prior to the first cytotoxic treatment.
Group 3: Received intra-peritoneal injection, containing 50 mg foscarnet/kilogram bodyweight, in sterile water on each of the three days following cytotoxic treatment.
Group 4: Received intra-peritoneal injection, containing 50 mg foscarnet/kilogram bodyweight, in sterile water, 5 minutes prior to the first cytotoxic treatment. Received one further identical injection on each of the three days following cytotoxic treatment.
Group 5: Vehicle control. Received intra-peritoneal injection, of sterile water, one hour prior to the first cytotoxic treatment. Received one further identical injection on each of the days following cytotoxic treatment.
Group 6: Vehicle control. Received intra-peritoneal injection, of sterile water, one hour prior to the first cytotoxic treatment.
Group 7: Vehicle control. Received intra-peritoneal injection, of sterile water, on each of the three days following cytotoxic treatment.
Group 8: Vehicle control. Received intra-peritoneal injection, of sterile water, 5 minutes prior to the first cytotoxic treatment. Received one further identical injection on each of the three days following cytotoxic treatment.

On the fourth day after treatment the animals of each group were killed, and intestinal tissue harvested for histological analysis. Tissue sections were stained with haemotoxylin and eosin, and were analysed for the presence of regenerating intestinal crypts. Regenerating intestinal crypts are derived from one or more surviving clonogenic stem cells, whereas sterilised crypts (containing no surviving stem cells) disappear within two days of irradiation. The number of regenerating crypts per intestinal circumference was counted, and the mean crypt width measured. Scores for each animal were then corrected to account for the probability of preferentially scoring larger crypts according to the following equation:

$$Corrected number = \frac{average width of untreated crypts}{average width of treated crypts} \times number of crypts$$

$$For each treatment a Protection Factor was calculated using the following equation \text{:} \frac{corrected crypts / circumference treated}{corrected crypts / circumference untreated ( vehicle )}$$

The results are shown in Tables 8 and 9 below, and illustrate that the treatments administered to both Groups 1, 2, 3 and 4 resulted in increased numbers of intestinal crypts in the experimental animals after exposure to 5-Fluorouracil. This indicates that the treatments protected intestinal epithelium clonogenic stem cells since they are, by definition, the only cells capable of initiating such regeneration. The intestinal epithelium of mice from group 1 contained nearly 50% more crypts after 50 mg/kg foscarnet treatment (compared to that of mice treated with vehicle alone), indicating that at least 50% more small intestinal clonogenic stem cells survived.

Since each clonogenic stern cell is able produce an exponential number of daughter cells this 50% increase in survival will, after cell expansion, cause a large increase in epithelial cellularity. Such cell survival and expansion can reduce diarrhoeas well as ulceration (mucositis) and other related conditions.

TABLE 8

| Treatment | | | | |
|---|---|---|---|---|
| 400 mg/kg | | | | corrected |
| 5FU x2 6 | mouse | no. crypts/ | crypt | crypts/ |
| hours apart plus: | no. | circumference | width/um | circumference |
| 50 mg | 1 | 16.00 | 29.37 | 17.28 |
| foscarnet/kg - | 2 | 35.70 | 33.63 | 33.67 |
| 1 hr, D1, D2, D3 | 3 | 32.50 | 34.46 | 29.92 |
| | 4 | 34.30 | 34.13 | 31.88 |
| | 5 | 24.30 | 32.51 | 23.71 |
| | 6 | 44.90 | 33.83 | 42.10 |
| | Ave | 31.28 | 32.99 | 29.76 |
| 50 mg | 1 | 42.50 | 38.42 | 35.09 |
| foscarnet/kg - | 2 | 16.20 | 31.11 | 16.52 |
| 1 hr | 3 | 24.70 | 34.22 | 22.90 |
| | 4 | 32.80 | 34.21 | 30.41 |
| | 5 | 11.70 | 32.90 | 11.28 |
| | 6 | 19.80 | 31.39 | 20.01 |
| | Ave | 24.62 | 33.71 | 22.70 |
| 50 mg | 1 | 14.10 | 32.56 | 13.74 |
| foscarnet/kg | 2 | 8.50 | 28.88 | 9.34 |
| D1, D2, D3 | 3 | 35.50 | 31.39 | 35.87 |
| | 4 | 24.10 | 33.06 | 23.12 |
| | 5 | 19.20 | 28.39 | 21.45 |
| | 6 | 16.10 | 33.74 | 15.14 |
| | Ave | 19.58 | 31.34 | 19.78 |
| 50 mg | 1 | 13.20 | 32.18 | 13.01 |
| foscarnet/kg - | 2 | 46.50 | 37.12 | 39.74 |
| 5 minutes, D1, | 3 | 40.10 | 32.92 | 38.64 |
| D2, D3 | 4 | 16.30 | 32.76 | 15.78 |
| | 5 | 24.30 | 31.60 | 24.39 |
| | 6 | 21.40 | 35.47 | 19.14 |
| | Ave | 26.97 | 33.68 | 25.12 |
| Vehicle - | 1 | 35.80 | 37.50 | 30.28 |
| 1 hr, D1, D2, D3 | 2 | 9.50 | 31.92 | 9.44 |
| | 3 | 8.50 | 30.73 | 8.77 |
| | 4 | 13.60 | 34.47 | 12.51 |
| | 5 | 13.60 | 29.11 | 14.82 |
| | 6 | 36.10 | 33.23 | 34.46 |
| | Ave | 19.52 | 32.83 | 18.38 |
| Vehicle - | 1 | 21.30 | 32.27 | 20.94 |
| 1 hr | 2 | 17.20 | 32.20 | 16.94 |
| | 3 | 23.20 | 31.62 | 23.27 |
| | 4 | 25.50 | 32.31 | 25.03 |
| | 5 | 25.40 | 31.60 | 25.50 |
| | 6 | 10.70 | 34.78 | 9.76 |
| | Ave | 20.55 | 32.46 | 20.24 |
| Vehicle | 1 | 9.10 | 28.56 | 10.11 |
| D1, D2, D3 | 2 | 30.60 | 31.90 | 30.43 |
| | 3 | 21.40 | 32.01 | 21.21 |
| | 4 | 5.90 | 33.35 | 5.61 |
| | 5 | 15.60 | 30.47 | 16.24 |
| | 6 | 22.90 | 33.48 | 21.70 |
| | Ave | 17.58 | 31.63 | 17.55 |
| Vehicle - | 1 | 23.20 | 32.70 | 22.50 |
| 5 minutes, | 2 | 19.60 | 32.06 | 19.39 |
| D1, D2, D3 | 3 | 13.20 | 27.92 | 15.00 |
| | 4 | 24.30 | 32.40 | 23.79 |
| | 5 | 28.20 | 32.72 | 27.34 |
| | 6 | 28.40 | 33.00 | 27.30 |
| | Ave | 22.82 | 31.80 | 22.55 |

TABLE 9

| Treatment | Protection Factor |
|---|---|
| 50 mg foscarnet/kg 1 hr prior to 1^{st} dose of 5- | 1.62 |
| Flurouracil (400 mg/kg x2 6 hrs apart) and D1, D2 | |
| & D3 | |
| 50 mg foscarnet/kg 1 hr prior to 1^{st} dose of 5- | 1.12 |
| Flurouracil (400 mg/kg x2 6 hrs apart) | |
| 50 mg foscarnet/kg D1, D2, D3 following 5- | 1.13 |
| Flurouracil (400 mg/kg x2 6 hrs apart) | |
| 50 mg foscarnet/kg 5 minutes prior to 1^{st} dose of | 1.11 |
| 5-Flurouracil (400 mg/kg x2 6 hrs apart) and D1, | |
| D2 & D3 | |

### Example 8

### Epithelial Cell Kinetic Evaluation Following Oral Administration of Foscarnet (Phosphonoformic Acid Trisodium Salt Hexahydrate)

Intestinal epithelial cell stimulation following an oral application of foscarnet (phosphonoformic acid trisodium salt hexahydrate) was illustrated by the following experiment.

Two experimental groups, each of three mice, were established as set out below:

Group 1: Received oral gavage, containing 500 mg foscarnet/kilogram bodyweight, in sterile water, once a day for 4 days. On the fifth day the animals received a pulse of 10 mg Bromodeoxyuridine (BrdUrd) and 40 minutes later were killed and small intestine and kidneys harvested for analysis.
Group 2: Received oral gavage of sterile water, once a day for 4 days On the fifth day the animals received a pulse of 10 mg Bromodeoxyuridine and 40 minutes later were killed and small intestine and kidneys harvested for analysis.

It will be noted that the orally administered dose of foscarnet utilised in Example 9 was greater than the intraperitoneal injection-administered doses used in the preceding Examples. This demonstrated both that oral administration represents a suitable route by which foscarnet may be provided in order to influence epithelial cell activity, and also that relatively high doses of foscarnet may be tolerated without notable toxicity.

During the course of the experiments there were no deaths or signs of illness in any group and at the end of the treatment period neither of the groups showed overt signs of kidney damage judged by histological sectioning.

Slides were immunohistochemically labelled for BrdUrd and counterstained with thionin to assess intestinal epithelial cell kinetic changes with oral administration of foscarnet. To assess epithelial cell kinetic changes, 50 small intestinal crypts per animal were quantified on a cell positional basis for the number of positively labelled S-phase cells at each cell position in the crypt. Quantification starts at the bottom of the crypt (cell position 1) and continues up one side of the crypt assessing each cell in turn to the crypt villus junction.

The results of Example 8 are illustrated in FIG. 5, which shows BrdUrd labelling index (calculated as a percentage of the total number of cells) in intestinal crypt cells of foscarnet-treated and control-treated animals. FIG. 5 illustrates that foscarnet has a stimulating effect on intestinal crypt cells as compared to the vehicle control.

### Example 9

### In Vivo Protection of Oral Mucosa Following Radiation Damage

The ability of foscarnet (phosphonoformic acid trisodium salt hexahydrate) to prevent radiological damage to oral mucosa was illustrated by the following experiment.

The model of cytotoxic insult used was treatment with 20 Gy X-ray radiation (head only) at a dose rate of 0.7 Gy/minute. Such radiation treatment provides a model of radiotherapy administered to patients undergoing treatment for cancer

Experimental groups, each of four mice, were established as set out below:

Group 1: Received intra-peritoneal injection, containing 50 mg foscarnet/kilogram bodyweight, in sterile water, one hour prior to radiation. Received one further identical injection on each of the two days following radiation. On the third day after radiation the animals were killed, and oral tissue harvested for analysis.
Group 2: Received intra-peritoneal injection, containing 50 mg foscarnet/kilogram bodyweight, in sterile water, one hour prior to radiation. Received one further identical injection on each of the four days following radiation. On the fifth day after radiation the animals were killed, and oral tissue harvested for analysis.
Group 3: Received intra-peritoneal injection of sterile water, one hour prior to radiation. Received one further identical injection on each of the two days following radiation. On the third day after radiation the animals were killed, and oral tissue harvested for analysis.
Group 4: Received intra-peritoneal injection of sterile water, one hour prior to radiation. Received one further identical injection on each of the four days following radiation. On the fifth day after radiation the animals were killed, and oral tissue harvested for analysis.

Tissue sections were immunohistochemically labelled for Bromodeoxyuridine and counterstained with thionin. Using a Zeiss AxioHOME the number of bromodeoxyuridine labelled and unlabelled cells were assessed in both the basal and suprabasal layers of the ventral surface of the tongue. The area from the basal layer to the stratum corneum stratum granulosum interface was measured along with the length of the basal layer. This was performed in 5 consecutive areas 2 mm back from the tip of the tongue. From these measurements the damage that the radiation had caused to the tongue could be assessed as overall cellularity of the tongue or the total number of cells/unit area (mm²).

The results of Example 9 are illustrated in FIGS. 6 and 7.

FIG. 6 compares the change with time in the number of cells per unit area on the ventral surfaces of the tongues of foscarnet-treated and vehicle-treated animals. The results shown in FIG. 6 illustrate that the overall cellularity of the ventral tongue over time is increased by pre- and post radiation treatment with foscarnet, as opposed to with vehicle control (sterile water).

FIG. 7 compares the average number of extra cells present per unit area in the epithelium covering the ventral surface of the tongues of foscarnet-treated and vehicle-treated animals. The results show that the average number of cells/unit area is increased on treatment with foscarnet, as compared to vehicle (sterile water) control.

For both FIGS. 6 and 7, treatment is an injection of foscarnet (50 mg/kg bodyweight) or sterile water one hour prior to 20Gy (head only) irradiation and then once a day until samples are taken (last injection being 24 hours prior to cull). Time, shown on the X axes of both FIG. 6 and FIG. 7, is the number of days following radiation.

### Discussion.

The cumulative experimental data presented in Examples 1-9 clearly show that the inhibitor of phosphate transporter activity foscarnet effectively reduces epithelial damage.

Foscarnet is able to reduce both damage to the intestinal epithelium, and hence instances of diarrhoea, and also damage to the oral mucosa. Furthermore, since previous studies with epithelial protective agents such as keratinocyte growth factor (KGF) have shown that protection of intestinal epithelia is a strong indicator for efficacy in protecting oral mucosa and reducing alopecia (Booth, C., and Potten, C. S. (2000). Keratinocyte growth factor increases hair follicle survival following cytotoxic insult. J Invest Dermatol 114, 667-673; Farrell, C. L., Bready, J. V., Rex, K. L., Chen, J. N., DiPalma, C. R., Whitcomb, K. L., Yin, S., Hill, D. C., Wiemann, B., Starnes, C. O., et al. (1998). Keratinocyte growth factor protects mice from chemotherapy and radiation-induced gastrointestinal injury and mortality. Cancer Res 58, 933-939; Farrell, C. L., Rex, K. L., Chen, J. N., Bready, J. V., DiPalma, C. R., Kaufman, S. A., Rattan, A., Scully, S., and Lacey, D. L. (2002). The effects of keratinocyte growth factor in preclinical models of mucositis. Cell Prolif 35 Suppl 1, 78-85), the data presented in Examples 1-9 also indicate that inhibitors of phosphate transporter activity, such as foscarnet and related compounds, will protect a wide range of epithelia from non-viral damage.

### Example 10

### Formulations

Examples of different formulations which may be used in accordance with the invention were produced as follows.

The formulations are illustrated with reference to phosphonoformic acid, preferably used in the form of its tri-sodium salt, though it will be appreciated that such formulations are appropriate for other inhibitors of phosphate transporter activity.

### 10.1 Shampoo Compositions

| | Amount | |
|---|---|---|
| Component: | (parts by weight) | |
| Ammonium Laureth Sulfate | 25 | g |
| Ammonium Lauryl Sulfate | 16.666 | g |
| Guar Hydroxypropyltrimonium chloride | 0.833 | g |
| Phosphono-formic acid | 0.2-20 | g |
| 1-decene homopolymer | 0.42 | |
| Trimethylpropane capyl caprylate | 0.42 | |
| Dimethicone | 2.08 | |
| Ethylene glycol distearate | 2.08 | |
| Cocamide MEA | 1.25 | |
| Cetyl alcohol | 1.875 | g |
| Propylene glycol | 0.208 | g |
| Methyl paraben | 0.208 | g |
| Water and minors | ad 100.0 | g |

### Method

Between one-third and all of the ammonium laureth sulfate (added as 25 wt % solution) is added to a jacketed mix tank and heated to about 60° C. to about 80° C. with slow agitation to form a surfactant solution. Cocamide MEA and the fatty alcohols are added to the tank and allowed to disperse. Salts (e.g. sodium chloride) and pH modifiers (e.g. citric acid, sodium citrate) are added to the tank and allowed to disperse. Ethylene glycol distearate (“EGDS”) is added to the mixing vessel and allowed to melt. After the EGDS is melted and dispersed, preservative (methyl paraben) is added to the surfactant solution. The resulting mixture is cooled to about 25° C. to about 40° C. and collected in a finishing tank. As a result of this cooling step, the EGDS crystallizes to form a crystalline network in the product. The remainder of the ammonium laureth sulfate and other components, including the silicone and phosphonoformic acid, are added to the finishing tank with agitation to ensure a homogeneous mixture. Cationic polymer is dispersed in water as an about 0.1% to about 10% aqueous solution and then added to the final mix. Once all components have been added, additional viscosity and pH modifiers may be added, as needed, to the mixture to adjust product viscosity and pH to the extent desired.

### 10.2 Foam for Application to Scalp

| Component: | Amount | |
|---|---|---|
| Phosphonoformic acid | 0.2-20 | g |
| Cetyl Alcohol BP | 1.10 | g |
| Octadecan-1-ol BP 0.50 | 1.10 | g |
| Polysorbate 60 BP | 0.40 | g |
| Ethanol | 57.79 | g |
| Propylene Glycol BP | 2.00 | g |
| Citric Acid Anhydrous BP | 0.073 | g |
| Potassium Citrate | 0.027 | g |
| Butane/Propane | 4.30 | g |
| Water and minors | ad 100.0 | g |

Method Cetyl alcohol (HYFATOL 1698, Efkay Chemicals Limited, London), octadecan-1-ol (HYFATOL 1898, Efkay Chemicals Limited, London), Polysorbate 60 (CRILLET 3, Croda Chemicals, North Humberside) and ethanol in the correct proportions are mixed and heated to about 45° C., with continuous stirring until the mix becomes clear. Phosphonoformic acid is slowly transferred into the mix, again with continuous stirring until the mix becomes clear. (Alcoholic Phase)

Purified water is separately heated to 45° C. and anhydrous citric acid BP and potassium citrate BP transferred to the water, with continuous stirring until dissolved. (Aqueous Phase)

The Alcoholic and Aqueous phases are each filtered through 75 micron screens and the required weights filled into a can (aluminium, epoxy lined) at room temperature. After attaching a valve, the butane/propane propellant (Propellant P70) is added to the mix in the can to the required weight, and an actuator added to the valve.

The composition, on being sprayed from the can onto the skin, produces a thermophobic foam which breaks down under heating from the skin to release the active compound to the epidermis.

### 10.3 Aerosol for Inhalation

| | | |
|---|---|---|
| Phosphonoformic acid (as its trisodium salt) | 1.00 | g |
| Miglyol ® | 0.20 | g |
| Frigen ® 11/12/13/14 | ad 100.0 | g |

### 10.4 Tablets

| Each tablet contains: | |
|---|---|
| Phosphonoformic acid (as its trisodium salt) | 20.0 mg |
| Maize starch | 25.0 mg |
| Lactose | 190 mg |
| Gelatin | 1.5 mg |
| Talc | 12.0 mg |
| Magnesium sterate | 1.5 mg |
| | 250 mg |

### 10.5 Suppositories

| Each suppository contains: | | |
|---|---|---|
| Phosphonoformic acid (as its trisodium salt) | 20.0 | mg |
| Ascorbyl palmitate | 1.0 | mg |
| Suppository base (imhausen H or Witepsol ® H) | ad 2000 | mg |

### 10.6 Syrup (I)

| | | |
|---|---|---|
| Phosphonoformic acid (as its trisodium salt) | 0.200 | g |
| Liquid glucose | 30.0 | g |
| Sucrose | 50.0 | g |
| Ascorbic acid | 0.1 | g |
| Sodium pyrosulfite | 0.01 | g |
| Disodium edetate | 0.01 | g |
| Orange essence | 0.025 | g |
| Certified colour | 0.015 | g |
| Purified water | ad 100.0 | g |

### 10.7 Injection Solution

| | | |
|---|---|---|
| Phosphonoformic acid (as its trisodium salt) | 0.500 | mg |
| Sodium pyrosulfite | 0.500 | mg |
| Disodium edetate | 0.100 | mg |
| Sodium chloride | 8.500 | mg |
| Sterile water for injection | ad 1.00 | ml |

### 10.8 Inhalation Solution

| | | |
|---|---|---|
| Phosphonoformic acid (as its trisodium salt) | 5.00 | g |
| Sodium pyrosulfite | 0.10 | g |
| Disodium edetate | 0.10 | g |
| Sodium chloride | 0.85 | g |
| Purified water | ad 100 | ml |

### 10.9 Sublingual Tablets

| | |
|---|---|
| Phosphonoformic acid (as its trisodium salt) | 5.0 mg |
| Lactose | 85.0 mg |
| Talc | 5.0 mg |
| Agar | 5.0 mg |
| | 100.0 mg |

### 10.10 Drops (I)

| | | |
|---|---|---|
| Phosphonoformic acid (as its trisodium salt) | 2.00 | g |
| Ascorbic acid | 1.00 | g |
| Sodium pyrosulfite | 0.10 | g |
| Disodium edetate | 0.10 | g |
| Liquid glucose | 50.00 | g |
| Absolute alcohol | 10.00 | g |
| Purified water | ad 100 | ml |

### 10.11 Syrup (II)

| | | |
|---|---|---|
| Phosphonoformic acid (as its trisodium salt) | 0.200 | g |
| Liquid glucose | 30.0 | g |
| Sucrose | 50.0 | g |
| Ascorbic acid | 0.1 | g |
| Disodium edetate | 0.01 | g |
| Orange essence with solubilizer | 0.25 | g |
| Hydrochloric acid to pH 6.0-6.5 | | |
| Purified water | ad 100.0 | g |

### 10.12 Solution for Injection

| | | |
|---|---|---|
| Phosphonoformic acid (as its trisodium salt) | 0.500 | mg |
| Disodium edetate | 0.100 | mg |
| Sodium chloride | 8.500 | mg |
| Hydrochloric acid to pH 6.0-7.0 | | |
| Sterile water for injection | ad 1.00 | ml |

### 10.13 Solution for Inhalation

| | | |
|---|---|---|
| Phosphonoformic acid (as its trisodium salt) | 5.00 | g |
| Disodium edetate | 0.10 | g |
| Sodium chloride | 0.85 | g |
| Hydrochloric acid to pH 6.0-6.9 | | |
| Purified water | ad 100 | ml |

### 10.14 Drops (II)

| | | |
|---|---|---|
| Phosphonoformic acid (as its trisodium salt) | 2.00 | g |
| Citric acid | 1.00 | g |
| Disodium edetate | 0.10 | g |
| Liquid glucose | 50.00 | g |
| Ethanol (95%) | 10.00 | g |
| Sodium hydroxide and hydrochloric acid to pH 6.2-6.8 | | |
| Purified water | ad 100 | ml |

### 10.15 Solution for Topical Use

| | | |
|---|---|---|
| Phosphonoformic acid (as its trisodium salt) | 2.00 | g |
| Isopropanol | 38.0 | g |
| Glycerol | 13.6 | g |
| Hydrochloric acid to pH 5.0-7.0 | | |
| Purified water | ad 100.0 | g |

### 10.16 Jelly

| | | |
|---|---|---|
| Phosphonoformic acid (as its trisodium salt) | 4.0 | g |
| Methocel ® | 4.0 | g |
| Methyl paraoxybenzoate | 0.12 | g |
| Propyl paraoxybenzoate | 0.05 | g |
| Sodium hydroxide and hydrochloric acid to pH 6.7 | | |
| Distilled water | ad 100 | ml |

### 10.17 Ointment (I)

| | | |
|---|---|---|
| Phosphonoformic acid (as its trisodium salt) | 2.5 | g |
| Cetyltrimethylammonium bromide | 0.6 | g |
| Stearyl alcohol | 2.25 | g |
| Cetanol | 6.75 | g |
| Liquid paraffine | 17.0 | g |
| Glycerol | 12.0 | g |
| Hydrochloric acid to pH 6.5 | | |
| Distilled water | ad 100.0 | g |

Preparations containing 0.2, 0.5, 1.0 and 2.0 g of phosphonoformic acid trisodium salt have also been prepared.

### 10.18 Ointment (II)

| | | |
|---|---|---|
| Phosphonoformic acid (as its trisodium salt) | 2.5 | g |
| Polyethylene glycol 1500 | 5.0 | g |
| Polyethylene glycol 4000 | 15 | g |
| Polyethylene glycol | ad 100 | g |

### 10.19 Ointment (III)

| | | |
|---|---|---|
| Phosphonoformic acid (as its trisodiurn salt) | 3.0 | g |
| Sorbitan monoleate | 5.0 | g |
| Petrolatum | ad.100 | g |

### 10.20 Gastric Juice-Resistant Tablets

Tablets, as described above, are coated with an enteric coating solution with the following composition.

| | | |
|---|---|---|
| Cellulose acetate phtalate | 120.0 | g |
| Polyethylene glycol | 30.0 | g |
| Sorbitan monoleate | 10.0 | g |
| Ethanol (95%) | 450.0 | ml |
| Acetone | q.s ad 1000.0 | ml |

The coating is carried out by a pouring procedure in a conventional coating pan or by spraying in a pan spray tablet coater.

1. The use of an inhibitor of phosphate transporter activity for the manufacture of a medicament for the prevention and/or treatment of non-viral damage to an epithelium, or of a condition caused or characterised by such damage.

2. The use according to claim 1, wherein the inhibitor of phosphate transporter activity is a phosphono-carboxylic acid, or a pharmaceutically acceptable derivative thereof.

3. The use according to claim 2, wherein the phospho-carboxylic acid is of the formula R¹R²P(O)-Lₙ-CO₂H, or a salt or ester thereof, wherein n is 0 or 1, R¹ and R² are the same or different and each is hydroxy or an ester residue; and L is a hydrocarbon group having from 1 to 8 carbon atoms.

4. The use according to claim 3, wherein the phospho-carboxylic acid is phosphonoformic acid or phosphonoacetic acid.

5. The use according to any of claims 2 to 4, wherein the pharmaceutically acceptable derivative is a salt or ester of the acid.

6. The use according to claim 4, wherein the pharmaceutically acceptable derivative is an alkali metal salt of phosphonoacetic acid or phosphonoformic acid.

7. The use according to claim 6, wherein the alkali metal salt is the sodium salt.

8. The use according to claim 7, wherein the sodium salt is the trisodium salt.

9. The use according to claim 8, wherein the trisodium salt is phosphonoformic acid trisodium salt.

10. The use according to any of claims 2 to 4, wherein the derivative is an amine or quaternary ammonium salt.

11. The use according to claim 1, wherein the inhibitor of phosphate transporter activity is a phosphatonin.

12. The use according to claim 11, wherein the phosphatonin is fibroblast growth factor 23 (FGF23).

13. The use according to claim 11, wherein the phosphatonin is frizzled-related protein 4 (FPF4).

14. The use according to any preceding claim, wherein the inhibitor of phosphate transporter activity is an inhibitor of sodium-dependent phosphate transporter activity.

15. The use according to claim 14, wherein the inhibitor of phosphate transporter activity is inhibitor of type III sodium-dependent phosphate transporter activity.

16. The use according to any preceding claim, wherein the damage is to clonogenic stem cells of the epithelium.

17. The use according to any preceding claim, wherein the epithelium is a digestive epithelium.

18. The use according to claim 17, wherein the damage is manifested in a condition selected from the group comprising mucositis, diarrhoea, colitis, ulcers, surgical or accidental wounds and reactive diseases such as inflammatory bowel disease.

19. The use according to any one of claims 1 to 16, wherein the epithelium is the epithelium of the scalp.

20. The use according to any preceding claim, wherein the non-viral damage is caused by a cancer therapy.

21. The use according to claim 20, wherein the non-viral damage is caused by chemotherapy.

22. The use according to claim 20, wherein the non-viral damage is caused by radiotherapy.

23. The use according to any of claims 1 to 19, wherein the non-viral damage is caused by microbial infection.

24. The use according to any of claims 1 to 19, wherein the non-viral damage is caused by reactive diseases such as inflammatory bowel disease.

25. The use according to any preceding claim, wherein the medicament is formulated for injection.

26. The use according to claim any of claims 1 to 24, wherein the medicament is formulated for oral administration.

27. The use according to any of claims 1 to 24, wherein the medicament is formulated for rectal administration.

28. The use according to any preceding claim, wherein the medicament is formulated for systemic administration.

29. The use according to any of claims 1 to 27, wherein the medicament is formulated for topical application.

30. The use according to claim 29, wherein the medicament is formulated as a shampoo.

31. The use according to any preceding claim, wherein the medicament is formulated for use in combination with a chemotherapeutic compound.

32. The use according to claim 31, wherein the medicament comprises the admixture of the inhibitor of phosphate transporter activity and the chemotherapeutic compound.

33. The use according to claim 31, wherein the inhibitor of phosphate transporter activity and the chemotherapeutic are provided in separate dosage forms.

34. The use according to any one of claims 31 to 33, wherein the chemotherapeutic compound is fluorouracil.

35. The use of a compound selected from the group comprising phosphonoacetic acid and phosphonoformic acid, and pharmaceutically acceptable derivatives thereof, for the manufacture of a medicament for the prevention and/or treatment of diarrhoea and/or mucositis caused by radiotherapy and/or by chemotherapy.

36. The use of a compound selected from the group comprising phosphonoacetic acid and phosphonoformic acid, and pharmaceutically acceptable derivatives thereof, for the manufacture of a medicament for the prevention and/or treatment of diarrhoea caused by non-viral microbial infection.

37. A shampoo composition comprising an inhibitor of phosphate transporter activity and at least one surface active agent suitable for shampooing hair.

38. A shampoo composition according to claim 37, wherein the inhibitor of phosphate transporter activity is a phosphono-carboxylic acid, or a pharmaceutically acceptable derivative thereof.

39. A shampoo according to claim 38, wherein the phosphono-carboxylic acid compound is the acetic or formic form.

40. The use of phosphono-carboxylic acid, or a pharmaceutically acceptable derivative thereof, for the manufacture of a medicament for the prevention and/or treatment of non-viral damage to an epithelium, or of a condition caused or characterised by such damage.

41. The use according to claim 40, wherein the phosphono-carboxylic acid or derivative is an acid or derivative as considered in any of claims 3 to 10.
